(19) United States
(12) Patent Application Publication
(10) Pub. No.: US 2026/0242856 A1
(43) Pub. Date: Aug. 20, 2026

(54) Diagnostic Platform

(71) Applicants: Brian Michael Coyle, Canyon, CA (US); Alys Larsen, Canyon, CA (US)

(72) Inventors: Brian Michael Coyle, Canyon, CA (US); Alys Larsen, Canyon, CA (US)

(21) Appl. No.: 19/643,289

(22) Filed: Apr. 9, 2026

(51) Int. Cl. C12Q 1/6844; C12Q 1/00; C12Q 1/25; C12Q 1/34; C12Q 1/6876

(52) U.S. Cl. CPC C12Q 1/6844; C12Q 1/001; C12Q 1/25; C12Q 1/34; C12Q 1/6876; C12Y 301/00; C12Q 2600/112; C12Q 2600/154; C12Q 2600/16

(57) ABSTRACT

A diagnostic platform comprises a fluid-accessible sampling assembly configured to capture, concentrate, and detect biomarkers from saliva, gingival crevicular fluid, or other oral fluids for disease screening, diagnosis, and monitoring. The platform includes a porous or composite sampling matrix retained within a fluid-permeable enclosure and is organized around four core module families: a capture-configuration module with heterogeneous material architecture providing spatially distinct capture regions that selectively bind distinct biomarkers; a transport or recruitment module biasing fluid and biomarker movement through capillary, electrostatic, and pore-structure effects; an integrated signal-generation module enabling colorimetric, fluorescent, electrochemical, or optical detection at the sampling assembly; and a fractionated recovery-flow module for staged, enriched analyte extraction. The platform achieves concentration factors of 10² to 10⁵, enabling multiplex detection of low-abundance biomarkers including proteins, nucleic acids, extracellular vesicles, and metabolites relevant to infectious disease, cancer, diabetes, cardiovascular disease, and inflammatory disorders.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The present disclosure relates to sampling matrices, sampling assemblies, and related systems and processes configured to capture, concentrate, recover, and optionally detect one or more biomarkers from saliva, saliva mixed with gingival crevicular fluid (GCF), or other oral fluids for use in disease screening, diagnosis, monitoring, surveillance, therapeutic assessment, or related applications

### Description of Related Technology

Oral fluids contain a wide range of biomarkers, proteins, antibodies, antigens, nucleic acids, extracellular vesicles, glycoproteins, metabolites, and microorganisms, associated with oral and systemic physiology and disease.

Because oral-fluid sampling is non-invasive and requires no venipuncture, it has long been regarded as an attractive alternative or complement to blood-based testing for home use, point-of-care testing, serial monitoring, pediatric sampling, and low-resource screening.

Despite these advantages, oral-fluid diagnostics remain limited by the physical and biochemical nature of saliva. Many clinically relevant biomarkers are present at concentrations substantially lower than in blood or tissue, and levels may vary widely with salivary flow rate, hydration state, stimulation conditions, collection method, oral health, mucosal permeability, gingival inflammation, time of day, food intake, and medication use. Biomarkers may also be distributed across distinct oral-fluid compartments, whole saliva, glandular secretions, GCF, mucosal transudates, cellular debris, and extracellular vesicles, complicating uniform recovery.

Conventional collection approaches, passive drool, absorbent swabs, sponges, pads, strips, or simple receptacles, can collect fluid but do not selectively enrich target biomolecules, preserve diagnostically useful relative concentrations across analyte classes, or provide robust control over sampling volume, transport, capture, release, or recovery.

These deficiencies are compounded by fouling: salivary mucins, glycoproteins, particulate matter, and nonspecific proteins can mask ligands, obstruct pores, alter interfacial transport, and reduce analyte accessibility to capture surfaces. Devices also typically lack mechanisms for fluid conditioning before capture, selective routing of analyte classes, or exploitation of charge, capillary, affinity, or size-based transport effects.

The diversity of clinically relevant biomarker classes presents a further challenge. Antibodies, antigens, nucleic acids, vesicles, glycoproteins, metabolites, and mucin-associated proteins may each require distinct local environments for effective capture, retention, release, and detection; architectures optimized for one class may perform poorly for another. Existing systems generally do not provide a configurable architecture capable of accommodating these differing behaviors through spatial organization, material heterogeneity, or staged transport and release. Post-capture handling is similarly limited: analytes may be released inefficiently or into poorly controlled volumes, and devices often lack mechanisms for staged recovery, fraction-directed detection, or controlled transfer of enriched fractions to a readout subsystem.

Collection and capture alone do not yield clinically meaningful results unless signals are interpreted in context. Raw oral-fluid signals may be distorted by dilution, variable contact time, recovery volume, hydration state, or subject-to-subject variation. Multiplex systems require normalization, analyte-ratio analysis, signal weighting, baseline and longitudinal comparison, or threshold evaluation to produce outputs such as classifications, risk scores, inflammatory indices, or treatment-response assessments. Existing devices rarely integrate such processing with the upstream sampling and concentration architecture. These deficiencies are especially pronounced in subjects with xerostomia, reduced salivary flow, altered GCF production, oral inflammation, or medication-related dryness, where passive collection may fail to obtain sufficient diagnostic material or may produce results that reflect sampling conditions more than disease status.

Accordingly, there remains a need for diagnostic platforms that actively engage the oral environment to contact larger fluid volumes, condition and route fluid before capture, localize analyte classes within spatially distinct capture territories, exploit affinity and transport effects to enrich target biomarkers, release and recover enriched fractions in a controlled manner, and integrate signal generation and computational interpretation into a unified workflow, adaptable across infectious, inflammatory, autoimmune, oncologic, metabolic, cardiovascular, neurodegenerative, epigenetic, and related diagnostic applications.

### BRIEF SUMMARY OF THE INVENTION

The diagnostic platform includes a sampling subsystem comprising one or more fluid-accessible sampling matrices configured to receive biological fluid and to capture, localize, partition, retain, release, or otherwise interact with one or more biomarkers. The sampling matrix may comprise porous, swellable, fibrous, scaffold-like, inorganic, organic, hybrid, or composite materials capable of supporting diagnostic capture and transport functions.

The platform includes at least one of four core module families:
(A) capture-configuration module
(B) transport or recruitment module
(C) integrated signal generation module
(D) fractionated recovery-flow module
A. Capture performance depends not only on affinity and ligand chemistry, but on how capture mechanisms are spatially deployed within the sampling matrix, including density distribution, pore architecture, material composition, microdomain organization, and alignment relative to fluid entry pathways. These configurations may operate independently of specific ligand identity and may involve heterogeneous material regions.

B. Transport of biomarkers may occur through fluid migration within the sampling matrix and through recruitment of biomarker-enriched fluid toward the sampling subsystem from adjacent regions. Such transport and recruitment may be influenced by capillary geometry, pore structure, electrostatic effects, material interfaces, deformation-responsive properties, or their combinations thereof.

C. Detection may occur at the sampling subsystem, including within the sampling matrix or at the fluid-permeable enclosure, and may generate colorimetric, fluorescent, electrochemical, optical, mechanical, or other detectable signals corresponding to one or more biomarkers.

D. Fractionated recovery flow may be configured such that a selectively extracted fluid fraction from within the sampling matrix contains a higher relative concentration of one or more analytes than other fluid remaining within the matrix or subsequently recovered. Flow-resistance control, staged transfer, sequential extraction, or inter-region interaction may be used to shape recovery and co-recover multiple analytes.

### GENERAL PLATFORM ARCHITECTURE

The diagnostic platform includes a sampling assembly comprising a fluid-accessible sampling matrix retained by a fluid-permeable enclosure. The fluid-permeable enclosure includes at least one fluid-permeable region through which biological fluid may enter and exit during sampling, such that the sampling matrix contacts the biological fluid and supports one or more of biomarker capture, analyte partitioning, transport bias, signal generation, conditioning, or analyte recovery.

The sampling matrix may comprise one or more inorganic, organic, polymeric, hybrid, fibrous, scaffold-like, foam-like, or composite materials configured to provide fluid accessibility and diagnostic functionality as described herein.

The diagnostic platform includes at least one module selected from one of the four core module families (A, B, C, D).

The capture-configuration module comprises one or more spatially distinct capture regions within the sampling matrix. Capture regions may be arranged as surface-proximal zones, radial layers, segregated domains, parallel pathways, or cascaded zones, and may differ in ligand identity or density, pore geometry, material composition, crosslink density, charge distribution, micro-or nano-patterning, antifouling character, or combinations.

Heterogeneous material architectures, composed of materials positioned relative to one another, contribute to differential biomarker attraction, transport, retention, release, or enrichment independently of ligand identity. They may be combined with ligand-based capture architectures.

The transport or recruitment module comprises one or more structures positioned within, adjacent to, or upstream of the sampling matrix and configured to bias movement of biological fluid or biomarkers relative to other biological compounds. Such structures may include charged regions, pore-size gradients, capillary-accessible geometries, wicking elements, fluidic conduits, or their combinations. In embodiments, these structures alter the composition of biological fluid reaching one or more capture regions prior to binding by immobilized capture domains and/or bias movement of biological fluid or biomarkers into or through the sampling matrix.

The integrated signal-generation module comprises one or more signal-generating elements associated with the sampling assembly such that diagnostic signals are generated at the sampling matrix or at the fluid-permeable enclosure. Signal generation may occur by sensing elements integrated into or onto the sampling matrix, sensing elements integrated into or onto the fluid-permeable enclosure, or signal-generating elements introduced into contact with the sampling matrix after biomarker binding. Such structures may permit diagnostic signals to be generated at the sampling assembly without requiring extraction of the matrix fluid to a separate diagnostic device. The generated signals may be visually observable, electronically measured, wirelessly transmitted, or communicated to an external processor or display device.

The fractionated recovery-flow module comprises one or more structures configured to direct recovery of a selected fluid fraction from the sampling matrix. Such structures may include capillary channels, porous wicks, microfluidic pathways, reservoirs, waste regions, valves, or flow-resistance features. The module may be configured such that an initial recovered fraction is enriched in one or more biomarkers relative to later-recovered fractions.

Any one of the foregoing modules may be used alone or in combination with one or more other enhancement modules. Thus, the diagnostic platform is not limited to embodiments containing all modules, and in certain embodiments includes the sampling matrix-based sampling assembly together with only a single module, while in other embodiments two, three, or four modules are combined in a single platform.

### Platform-Level Concentration and Multiplex Utility

In certain embodiments, the diagnostic platform functions as a volumetric molecular integrator. Salivary biomarker levels may in some contexts be diluted relative to serum levels by factors on the order of 10⁻¹ to 10⁻³, while the sampling, capture localization, transport bias, and controlled recovery architectures of this invention may provide effective concentration factors on the order of 10² to 10⁵ by sampling larger biological fluid volumes and delivering smaller recovered fractions for analysis.

In certain embodiments, the platform is useful for multiplex detection of biomarkers in saliva or other dilute biological fluids, including proteins, antibodies, antigens, nucleic acids, extracellular vesicles, glycoproteins, metabolites, or combinations thereof. Volumetric concentration factors may be especially useful in multiplex embodiments directed to diabetes, liver disease, HIV, tuberculosis, inflammatory panels, cancer-associated biomarkers, or other multi-analyte diagnostic applications.

### Definition of Sampling Matrix

As used herein, the term “sampling matrix” (“matrix”) refers to a fluid-accessible structural medium configured to receive biological fluid and to support one or more of biomarker interaction, analyte partitioning, transport bias, signal generation, conditioning, regional capture configuration, deformation-responsive behavior, or analyte recovery. The diagnostic platform matrix may comprise any porous, swellable, fluid-accessible, or biologically compatible material configured to receive biological fluid, present one or more capture ligands or affinity domains, and preferentially localize, partition, retain, concentrate, or release one or more biomarkers or biomarker-containing fractions.

Matrices especially useful for oral or salivary use include hydrogel matrices, cellulose-based matrices, polyvinyl alcohol matrices, silk fibroin matrices, collagen-based matrices, and selected silica-based matrices. In embodiments, decellularized extracellular matrix materials, hydrocolloid-based matrices, polyurethane foams, liquid crystal elastomers, and other responsive or composite structures may be used where appropriately configured for biocompatibility, wet-state stability, ligand presentation, deformation tolerance, or their combinations. The sampling matrix may be implemented as a single material body or as a heterogeneous architecture comprising multiple material regions, layered structures, interpenetrating phases, particle-supported domains, or in combinations.

The sampling matrix may be a capture matrix configured to perform one or more of the functions otherwise described herein, including selective capture, transport bias, regional capture configuration, integrated signal generation, fractionated recovery flow, dynamic deformation-dependent transport, analyte partitioning, enrichment, or release, or combinations. The diagnostic platform encompasses a broad class of fluid-accessible biomarker-capture matrices capable of supporting the modular diagnostic functions.

### BRIEF DESCRIPTION OF THE DRAWINGS

FIG. 1 Representation of diagnostic platform.

FIG. 2 Sectional functional schematic diagram of a tuberculosis embodiment.

FIG. 3 Sectional functional schematic diagram of a cardiovascular embodiment.

FIG. 4 Fractionated recovery arrangement for a multiplex liver disease embodiment.

FIG. 5 Sectional functional schematic of a cardiovascular embodiment.

FIG. 6 Normalization flow diagram of a diabetes embodiment.

FIG. 7 Methylation-selective capture schematic diagram.

FIG. 8 Schematic of HIV biomarker-attracting fluid recruitment.

FIG. 9 Staged elution and fractionated-recovery schematic of an ovarian cancer embodiment.

FIG. 10 Computational logic workflow for inflammatory indices.

FIG. 11 Illustration of a sampling apparatus for a Sjogren's syndrome embodiment.

### DETAILED DESCRIPTION OF THE INVENTION

### Detailed Description of Matrix Materials

The matrices described herein may comprise any of the materials listed in Definition of Sampling Matrix. Each of the functional modules, including capture configuration, transport bias, signal generation, and fractionate recovery, may be implemented in association with any of the matrix materials described below, which serve as structural materials and provide biomarker-capture functions.

In certain embodiments, the matrix includes a hydrogel matrix. The hydrogel matrix may comprise a covalently, ionically, or a physically crosslinked hydrogel, a cryogel, interpenetrating polymer network, a composite hydrogel, particulate hydrogel, or combinations. The hydrogel matrix may comprise one or more polymers selected from polysaccharides, synthetic hydrophilic polymers, proteins, peptides, or combinations. Examples include agarose, alginate, carrageenan, hyaluronan, dextran, cellulose derivatives, chitosan, gelatin, collagen-containing hydrogel systems, polyvinyl alcohol, polyethylene glycol, polyacrylamide, or combinations.

The hydrogel matrix may provide a water-rich, deformable, and chemically functional environment suitable for biomarker contact, ligand presentation, analyte partitioning, and controlled release. Hydrogel matrices may be advantageous due to tunable mesh size, swelling behavior, charge density, optical accessibility, softness, and compatibility with immobilized ligands or embedded sensing elements. The hydrogel matrix may be configured to include surface-proximal capture zones, depth-distributed capture regions, charge-partitioning domains, particle-supported microdomains, or gradients of pore size, ligand density, hydration, or crosslink density.

In some embodiments the hydrogel matrix is combined with another material phase, including silica, cellulose, collagen, silk, hydrocolloid, or foam-like structural elements, to improve mechanical durability, transport control, wet-state stability, or regional differentiation of function.

Certain embodiments include a matrix that is a silica-based material, that may comprise silica gel, mesoporous silica, organosilica, silica-polymer composite material, silica-coated porous substrate, or combinations. The silica-based matrix may be configured as a monolith, porous bead, granular body, molded body, coated framework, or enclosure-retained porous insert. Silica-based matrices may be advantageous due to high internal surface area, tunable pore size, surface silanol chemistry, capacity for chemical functionalization, and relative chemical stability. The silica-based matrix may be modified with organic polymers, biocompatible coatings, surface ligands, hydrated polymer phases, or their combinations to increase softness, reduce brittleness, reduce mucosal irritation, increase wet-state compatibility, or improve biomarker accessibility. Indicator silica formulations containing cobalt salts or other undesired indicator agents may be excluded. Mesoporous and organosilica systems are especially attractive because they are readily functionalized and have already been explored in oral and transmucosal biomedical contexts.

Certain embodiments include a matrix that is a cellulose-based material, and may comprise cellulose paper, regenerated cellulose, cellulose acetate, bacterial cellulose, nanocellulose, cellulose sponge, cellulose fiber network, cellulose derivative gel, or combinations. The cellulose-based matrix provides a porous and hydrophilic scaffold that absorbs biological fluid and presents one or more affinity ligands, charge domains, responsive chemistries, or signaling elements. The cellulose-based matrix may be chemically modified to provide aldehyde, carboxyl, amine, boronate, click-reactive, or ion-exchange functionality, or other ligand-coupling chemistry. Cellulose-based matrices are particularly attractive because they are generally biocompatible, broadly used in food and medical contexts, readily fabricated in thin or compressible forms, and adaptable to oral fluid sampling and biosensing formats.

Certain embodiments include a matrix that is a polyvinyl alcohol (PVA) material, a biocompatible base polymer, and may comprise a PVA hydrogel, PVA cryogel, porous PVA network, PVA composite, or interpenetrating polymer network containing PVA. The PVA matrix may be physically crosslinked, freeze-thaw processed, or produced in a manner that reduces or avoids residual toxic crosslinking reagents. The PVA matrix may provide mechanical resilience, water retention, optical clarity, pore accessibility, or molecular-sieving behavior useful for oral diagnostic sampling. The PVA matrix may be combined with cellulose, alginate, gelatin, collagen, silica, or another polymeric or inorganic phase to modify transport, strength, ligand density, or wet-state stability.

Certain embodiments include a matrix that is a collagen-based material, and may comprise a collagen sponge, collagen gel, fibrous collagen scaffold, collagen composite, collagen-coated porous substrate, or collagen-containing interpenetrating network. Collagen provides a biologically compatible and hydrophilic environment for biomarker contact and ligand presentation. The collagen matrix may be crosslinked, reinforced, blended with another polymer, or enclosure-supported to improve dimensional stability, wet-state strength, or deformation tolerance in the oral cavity. Collagen may be used as a primary capture body, or may provide a surface or interface layer, or affinity-supporting phase over another porous scaffold. Collagen may benefit from reinforcement or combination with another matrix-forming material.

Certain embodiments include a matrix that is a silk fibroin material, and may comprise a silk sponge, porous silk scaffold, silk film, electrospun silk network, silk hydrogel, silk composite, or silk-coated support. Silk fibroin provides a combination of biocompatibility, mechanical robustness, flexible processing, and chemical functionality. Silk fibroin matrices may be biomarker-capture matrices, and may support immobilized ligands, stabilize captured biomolecules, resist rapid dissolution, or provide a deformable yet durable porous capture architecture, and may be adapted to sensing-related applications. Silk fibroin may be combined with another polymer, inorganic phase, or signaling system.

Certain embodiments include a matrix that is a decellularized extracellular material, configured as a sheet, powder-derived porous body, gelled matrix, coating, composite phase, or ligand-bearing interface. A decellularized extracellular matrix provides a biologically derived microenvironment containing proteinaceous or glycosaminoglycan-rich components that influence wetting, transport, affinity, or biomolecule stabilization. The decellularized extracellular matrix may be further purified, crosslinked, or blended, or be supported by another scaffold material in a composite, providing an affinity-supporting or tissue-compatible component.

Certain embodiments include a matrix that is hydrocolloid-based material, and may comprise one or more of alginate, pectin, carboxymethylcellulose, xanthan gum, carrageenan, gelatin, gellan gum, guar-derived materials, or their combinations. In embodiments, the hydrocolloid-based matrix provides hydration control, swelling capacity, mucoadhesion, softness, or oral compatibility. The hydrocolloid-based matrix may function as a primary matrix, or it may be used as a layer to condition a surface, regulate matrix hydration, or for added comfort over a scaffold.

Certain embodiments include a matrix that comprises a polyurethane foam or related polymeric foam. The polyurethane foam may be open-cell, semi-open-cell, reticulated, coated, surface-functionalized, composite-filled, or otherwise configured to provide fluid accessibility and mechanical resilience. The foam may provide compressibility, rebound, and high void volume suitable for mouth entry and repeated deformation. It may be coated or modified with one or more hydrophilic polymers, biocompatible layers, or capture ligands to reduce hydrophobicity and improve biomarker interaction. In some embodiments, the polyurethane material is a structural support rather than the principal biochemical capture. Polyurethane foams may be used where repeated compression is expected.

Certain embodiments include a matrix that comprises a liquid crystal elastomer or other ordered responsive elastomeric network. Such a matrix is configured to undergo deformation-responsive changes in porosity, anisotropy, permeability, or transport behavior under chewing, compression or other stimulus. The liquid crystal elastomer may be used for diagnostics by biocompatible formulation, enclosure retention, surface modification, composite construction, or isolation from direct mucosal exposure. Such materials provide a mechanically responsive transport substrate rather than a conventional passive absorbent matrix. Responsive elastomeric matrices may be used for dynamic transport modulation or mechanically induced signaling.

Certain embodiments include a matrix that comprises a composite of two or more materials selected from silica-based, cellulose-based, polyvinyl alcohol-based, collagen-based, silk fibroin-based, decellularized extracellular matrix-based, hydrocolloid-based, polyurethane-based, liquid crystal elastomer-based, and hydrogel-forming polymer-based materials. For example, a silica phase may provide surface area and ligand-coupling chemistry while a polymer phase provides softness and oral compatibility; a cellulose or foam scaffold may provide structural support while a hydrogel or hydrocolloid phase provides wet-state transport control; a collagen or extracellular matrix phase may provide a biologically interactive interface while a synthetic polymer phase provides durability. A composite matrix may be layered, interpenetrating, coated, impregnated, laminated, gradient-structured, or otherwise differentiated.

In certain embodiments, the matrix materials are configured for use in the oral cavity and are selected or modified to satisfy one or more criteria selected from biocompatibility, non-toxicity, low mucosal irritation, resistance to fragmentation or to excessive swelling, controlled deformability, ligand-coupling capacity, biomarker accessibility, retention within an enclosure, resistance to cyclical compression or to premature dissolution, and compatibility with integrated signaling or recovery-flow architectures. In some embodiments a material that is not ideal for direct oral exposure is nevertheless useful when coated, composited, enclosure-retained, softened, reinforced, or otherwise modified.

### Detailed Description of Modules

### A. Capture-Configuration Module

In certain embodiments, the diagnostic platform includes a capture-configuration module (configuration module) comprising a fluid-accessible sampling matrix (matrix) having two or more spatially distinct capture regions arranged in a defined spatial configuration. The capture regions are positioned so that biological fluid entering the matrix encounters different capture environments at different locations, depths, pathways, orientations, or stages of fluid penetration, rather than encountering equivalent capture functionality throughout. The configuration module thus defines a physically organized internal capture architecture rather than a substantially uniform bulk structure.

Capture regions may differ in one or more structural or chemical properties, including ligand identity, class, or density; pore size or pore-size distribution; crosslink density; swelling behavior; charge distribution; hydrophobicity or hydrophilicity; antifouling character; mesh size; stiffness; diffusional resistance; degradation capacity; material composition; or combinations. A first capture region may be configured to preferentially bind a first analyte class and a second to preferentially bind a second analyte class; alternatively, multiple regions may bind the same analyte under different transport, access, selectivity, or fouling conditions.

Capture regions may be arranged by depth relative to the external fluid-entry surface, comprising surface-proximal, intermediate, and deeper capture zones that permit early-contact capture, delayed-access capture, size-biased penetration, or sequential analyte interaction as fluid moves into the matrix. Regions may also be arranged as radial layers, shell-and-core structures, stacked lamellae, longitudinal segments, parallel lanes, discrete islands, nodules, channels, pockets, or dispersed microdomains. Separated domains may direct fluid entering from different parts of the enclosure into different capture territories; parallel capture pathways may differ in selectivity, permeability, or binding function.

The configuration module may include continuous, stepwise, or regionally segmented gradients of ligand density, pore size, charge density, crosslink density, hydration state, antifouling functionality, or material composition. Gradients may be oriented from exterior to interior, or laterally, circumferentially, radially, axially, or along a defined fluid pathway, biasing where a given biomarker is localized, retained, or enriched.

One or more capture regions may be configured primarily for target binding, while additional regions perform exclusion, conditioning, shielding, interference reduction, ionic conditioning, or size-selective prefiltration. For example, a surface or upstream region may have reduced nonspecific adsorption, altered ionic states, or size-based permeability, while a downstream or adjacent region has higher affinity for target biomarkers. The configuration module may thereby spatially separate conditioning and capture functions within a single matrix.

Capture microdomains may be formed by incorporating ligand-bearing particles, beads, nanostructures, or functionalized microcarriers, such as silica particles, polymer beads, hydrogel microparticles, magnetic particles, cellulose-derived particles, or composite carriers, into defined matrix regions. Particle groups sharing the same or different ligands may be deposited in spatially concentrated zones, generating localized regions of increased ligand density arranged in layered, radial, cascaded, or patterned configurations.

Binding agents immobilized in different matrix regions may include antibodies, antibody fragments, aptamers, peptides, proteins, receptors, lectins, nucleic acids, molecularly imprinted binding sites, charged polymers, affinity dyes, metal-chelating groups, boronate-containing groups, ion-exchange groups, host-guest binding groups, or combinations, forming a heterogeneous capture map throughout the matrix.

The configuration module may be configured so that analytes of different size, diffusivity, abundance, or binding kinetics localize in different spatial regions: larger or more slowly diffusing analytes captured near the surface, smaller or more rapidly diffusing analytes penetrating more deeply, and abundant background components intercepted in a first region while target biomarkers are retained in a second. This spatial arrangement increases effective target concentration in a selected region.

Fabrication methods include sequential deposition, layered casting, localized crosslinking, photopatterning, printing, extrusion, lamination, embedding of prefabricated subdomains, differential post-fabrication functionalization, location-specific ligand coupling, selective area shielding during modification, dehydration or rehydration of local areas, and composite assembly. The matrix may be a monolithic body with internally differentiated capture regions or comprise two or more joined portions each providing a distinct capture function.

Heterogeneous material regions contribute to differential capture behavior: a silica-based region may provide high surface area and ligand-coupling density; a polymeric or hydrogel region provides deformability and fluid conditioning; a cellulose or foam scaffold provides structural support while a swellable phase regulates transport; a collagen or extracellular-matrix-derived region provides a biologically interactive interface while a synthetic polymer phase provides mechanical durability. Such regions may interact sequentially, cooperatively, or through coupled or dynamic mechanisms, including non-linear forces, to influence biomarker localization, enrichment, retention, or release.

The configuration module enables selective localization and architectural control of capture behavior that cannot be accomplished in a homogeneous capture body. In certain embodiments the configuration module is implemented alone; in others, in combination with one or more of the transport or recruitment module, integrated signal-generation module, and fractionated recovery-flow module. Where cooperating with the fluid-permeable enclosure, one or more enclosure regions align with corresponding capture regions so that fluid entering through a first permeable region accesses a first capture region and fluid entering through a second region accesses a second, coordinating entry position, penetration path, and capture location.

### B. Transport or Recruitment Module

In certain embodiments, the diagnostic platform includes a transport or recruitment module (transport module) comprising one or more structures, regions, material properties, or forces configured to bias movement of biological fluid, biomarkers, or biomarker-containing fractions toward, into, through, within, or out of the matrix. The transport module establishes a preferential transport environment that alters how fluid components approach, enter, partition in, or contact capture regions, rather than relying on passive exposure.

The transport module may increase delivery of one or more target biomarkers to a selected capture region while reducing access of non-target components, interferents, fouling species, or high-abundance background constituents, with preferential recruitment and preferential exclusion occurring concurrently. Transport-biasing features may include charged or ion-selective regions, pore-size or mesh-size gradients, wettability gradients, capillary-active geometries, wicking elements, porous pathways, conduits, channels, reservoirs, prefocusing regions, depletion zones, absorbent regions, osmotic regions, electrostatic regions, hydrophilic or hydrophobic regions, affinity-guiding regions, or combinations, positioned within the matrix, at a matrix surface, in or on the enclosure, or upstream of the matrix.

Upstream conditioning regions may alter biological fluid composition before it reaches a primary capture region by delaying, excluding, diverting, sequestering, filtering, or partitioning cells, debris, mucins, aggregates, particulates, proteins, or other interferents while permitting passage of target biomarkers, establishing a pre-capture recruitment path that enriches the fluid presented to a downstream capture region.

Transport may be staged: a first region provides recruitment, preconcentration, electrostatic partitioning, or size-based selection; a second provides more selective or higher-affinity capture. Larger or more transport-limited biomarkers localize near surface-proximal regions; smaller or more rapidly diffusing biomarkers penetrate more deeply. The transport module may also stage handling of vesicles and vesicle-associated biomarkers, retaining intact vesicles at a first region and transferring vesicle-derived contents to a second.

Capillary-active structures draw biological fluid into contact with the matrix without external pumping. Selected wettability, pore geometry, pore radius, pore interconnectivity, and surface energy generate localized capillary pressure that advances fluid into or across the matrix, at the matrix surface, within the matrix body, within the enclosure, or in a coupled structure.

Electrostatic or ionic recruitment features, fixed-charge regions, ion-exchange or conditioning features, polyelectrolyte domains, Donnan-active regions, zwitterionic domains, or salt-responsive regions, create local microenvironments that recruit, partition, retain, retard, or exclude selected analytes. Fixed positive charge preferentially recruits negatively charged biomarkers; fixed negative charge preferentially recruits positively charged biomarkers. Despite ionic screening in biological fluids, local charge density, mesh size, and porosity can increase analyte encounters with an immobilized capture domain.

Biomarker enrichment may arise from combined hydraulic transport, electrostatic partitioning, and steric selectivity: capillary-driven imbibition draws fluid inward, electrostatic potential increases local concentration of oppositely charged analytes, and steric constraints slow or exclude competing macromolecules. Neutral species follow bulk flow streamlines; charged species experience additional electric-field bias, shifting concentration gradients dynamically within the matrix.

Transport properties may derive from material composition. The matrix may comprise polysaccharide-based hydrogels (agarose, alginate, carrageenan, hyaluronan, dextran, cellulose derivatives, chitosan, or combinations), cellulose-based scaffolds, PVA networks, collagen matrices, silk fibroin matrices, silica-based matrices, composite matrices, or responsive elastomeric structures, with transport governed by intrinsic properties including porosity, deformability, fixed-charge density, surface energy, and hydration behavior.

Dynamic mechanical loading contributes to transport bias. Cyclical compression and relaxation may alter pore geometry, interstitial fluid flux, and hydraulic permeability, increasing uptake of biomarkers, particularly larger or more transport-limited biomolecules, relative to static diffusion. Combined matrix deformation and interstitial fluid movement may generate strain-dependent partitioning, matrix-solute drag, or pumping-like effects that bias biomarker transport. Cyclic compression and decompression may render capillary driving forces time-varying and alter electrostatic partitioning through changes in local pore geometry, hydration state, or dielectric environment. The resulting transport behavior reflects non-linear or synergistic interactions among steric exclusion, binding kinetics, electrostatic partitioning, and hydraulic flow.

Mechanical energy imparted during use, such as mastication, may cause biomarker accumulation to approach an active, deformation-driven equilibrium rather than a passive diffusion equilibrium. During compression, biomarkers remain associated with the matrix while fluid is expelled and mixed with surrounding biological fluid; upon relaxation, the matrix reabsorbs fluid, enabling further biomarker recruitment. Successive deformation cycles progressively deplete surrounding fluid of one or more biomarkers, a process that differs from passive equilibrium systems governed by static partitioning and diffusion. In some embodiments, the surrounding fluid approaches a depletion-limited condition in which further extraction is reduced.

Enclosure-associated transport features may also bias fluid entry: regions of differing porosity, wettability, thickness, charge, or permeability in the enclosure condition fluid differently depending on entry location, influencing subsequent analyte movement, localization, and capture.

The transport module may operate passively through capillary action, diffusion, electrostatic bias, partition bias, or selective retardation, or may cooperate with externally applied pressure, compression, vibration, magnetic or electrical influence, centrifugal force, or imposed flow. Externally responsive features, swellable, pH-responsive, salt-responsive, thermoresponsive, or mechanically responsive regions, may further alter permeability or analyte transport.

The transport module may be used alone or in combination with the configuration module, signal module, and fractionation module, cooperating with heterogeneous capture architectures to deliver recruited analytes to selected internal regions. Collectively, it provides a biased transport architecture that conditions, directs, partitions, concentrates, or localizes biological fluid components relative to the matrix, increasing controlled analyte access to diagnostically useful capture regions.

### C. Integrated Signal-generation Module

In certain embodiments, the diagnostic platform includes an integrated signal-generation module (signal module) comprising one or more signal-generating elements functionally associated with the matrix and/or the enclosure, so that a diagnostically useful signal is generated at, in, on, or adjacent to the matrix without requiring extraction of biological fluid in the matrix to a separate analytical instrument or assay platform.

Signal generation may occur in response to biomarker binding, accumulation, partitioning, or transport into the matrix; biomarker-induced reaction; displacement of a reporter; activation of an indicator; cleavage of a substrate; conformational change of a sensing element; or changes in ionic environment, hydration state, swelling state, conductivity, impedance, capacitance, optical property (color, fluorescence, phosphorescence, luminescence, absorbance, reflectance, scattering), mechanical property, electrochemical property, or combinations.

Colorimetric or fluorescent reporter systems may be integrated with the matrix. Where the matrix is optically transmissive or sufficiently transparent, excitation light and emitted or reflected signal may be collected from within the matrix. Alternatively, an optically accessible region, window, thin surface layer, or patterned zone may facilitate optical readout. Affinity ligands (antibodies, antibody fragments, aptamers, or others) associated with capture regions may be labeled directly or indirectly with a signal-generating enzyme, fluorophore, nanoparticle, or other reporter. A sandwich assay format may be employed, with a captured biomarker contacted by a secondary labeled detection ligand; alternatively, a reporter system may be pre-positioned in or on the matrix prior to exposure to biological fluid.

After biomarker capture, an introduced substrate solution allows enzyme labels, such as horseradish peroxidase, alkaline phosphatase, glucose oxidase, or combinations, to catalyze conversion of a chromogenic or fluorogenic substrate into a detectable product. Signal generation may occur within a capture region, at a surface-proximal zone, in a thin adjacent detection layer, within the enclosure, or in a coupled structure. Signal intensity, area, spatial distribution, or rate of development may correlate with bound biomarker amount. In fluorescent embodiments, excitation light is directed into a defined region and emitted fluorescence collected from the same or opposite surface; signal magnitude may be determined from intensity, wavelength shift, spatial distribution, or time-dependent accumulation. The signal may be directly visually observable without instrumentation, as color change, pattern formation, zone development, line formation, symbol appearance, intensity shift, or opacity change, and read by visual inspection, optical reader, imaging device, or smartphone camera.

The matrix may be a site of diagnostic analysis, so that biological fluid in the matrix is analyzed without prior extraction from the matrix. The matrix may be inserted into a cartridge, enclosure, or other support structure after sampling, but analysis remains localized in the matrix rather than in an extracted fluid sample. The support structure provides physical treatments, reagent delivery, thermal control, containment, or readout. Diagnostic analysis of a hydrogel matrix may include hydrogel loop-mediated isothermal amplification, fluorescent hydrogel devices, hydrogel colorimetric sensing, and electrochemical hydrogel sensing. All of these diagnostic analysis methods may be used with non-hydrogel matrices, providing diagnostic analysis localized in or at the matrix.

Electrochemical sensing elements may also be associated with the matrix. Detection ligands or ligand-linked reporter systems generate an electrical response upon target biomarker interaction. Electrodes may be integrated in the matrix, embedded in the enclosure, positioned at an interface, or provided in an adjacent conductive structure, with planar, interdigitated, mesh-like, patterned, or distributed geometries corresponding to one or more capture regions. A redox-active ligand, redox-labeled antibody, redox-tagged aptamer, enzyme-linked affinity reagent, or similar construct may undergo a detectable electrochemical change upon target binding by altering spacing, orientation, steric accessibility, or local environment of a redox reporter relative to an electrode, producing a measurable current or voltage shift. Enzyme-assisted electrochemical detection may employ mediator molecules within the matrix or enclosure to facilitate electron transfer. Detection may occur while biomarkers remain bound within the matrix, after localized release within a defined region, or following interaction with a secondary ligand introduced after sampling.

Signal generation may be spatially localized: a first region generates a signal corresponding to a first analyte class and a second region to a second analyte class. In multiplex embodiments, separate electrodes, optical regions, or patterned domains correspond to different capture territories, preserving positional information associated with biomarker localization. The signal module may cooperate with the configuration module so that heterogeneous capture architectures yield regionally differentiated signals.

In aptamer-based embodiments, an aptamer immobilized in or near the matrix undergoes a conformational change upon binding its target, turning fluorescence or electrochemical signals on or off by altering redox label proximity to an electrode. Because signal arises from conformational change rather than bulk reagent development, such embodiments permit rapid, localized, and potentially reversible detection. Label-free sensing, via changes in impedance, capacitance, conductivity, field-effect characteristics, optical scattering, refractive index, or mechanical response, may alternatively indicate biomarker binding or accumulation.

Signal generation may occur under static conditions or during or after dynamic conditions including fluid influx, fluid efflux, compression, decompression, mastication, or cyclical deformation. Dynamic mechanical loading may alter analyte distribution, electrode proximity, hydration state, ionic strength, or capture-zone occupancy, modulating timing, magnitude, or spatial pattern of signal generation. Signal intensity or temporal profile may reflect non-linear interactions among transport bias, capture localization, deformation-induced permeability changes, and reporter activation.

The sampling assembly may include integrated conductive traces, antenna elements, optical components, photodetectors, transducers, or communication modules, transmitting signals via near-field communication, radiofrequency components, Bluetooth-capable elements, passive or active tags, processors, or memory elements to an external reader, processor, smartphone, display, or networked system. Computational elements may interpret diagnostic signals in view of volumetric concentration parameters, including biological fluid volume contacted during sampling, recovered fraction volume, estimated or measured concentration factor, multiplex analyte ratios, sampling duration, deformation cycles, elution volume, or hydration state, and may assess whether the capture and recovery architecture yields concentrated diagnostic fractions that restore or exceed serum-equivalent detectability for selected analytes.

The signal module may be employed alone or with the configuration module, transport module, and fractionation module. Transport bias may alter timing or intensity of signal generation; heterogeneous capture regions may generate distinct signals or portions of a composite signal; a biomarker-enriched fraction recovered from the matrix may be analyzed by a reporter integrated with or coupled to the sampling assembly.

### D. Fractionated Recovery-Flow Module

In certain embodiments, the diagnostic platform includes a fractionated recovery-flow module (fractionation module) configured to selectively recover a defined portion of fluid from the matrix such that the recovered fraction is enriched in one or more biomarkers relative to other fluid within the matrix.

Upon application of a transport mechanism to the matrix, which retains a volume of biological fluid following sample contact, the initial recovered fraction may contain a higher relative concentration of one or more biomarkers than later-recovered fluid, producing a diagnostically enriched fraction without chemical amplification. Enrichment arises from spatial capture architecture and staged release: capture regions localize biomarkers in surface-proximal, intermediate, or depth-distributed zones, and upon applying a transport mechanism, fluid associated with certain regions is released preferentially.

In certain embodiments, the first several microliters of recovered eluate contain elevated target biomarker concentrations; approximately 5-20 uL of initial eluate may be selectively directed toward a detection region while later-released fluid is diverted to waste. Capillary wicking may dehydrate a surface region of the matrix, alter local swelling state, and modify hydration shells surrounding proteins, modulating antibody-target affinity and promoting release; small pressure gradients from capillary pull may flex the matrix network and assist selective detachment of bound or partitioned species.

Release chemistry may be staged: pre-equilibration with a mild elution buffer initiates specific release equilibria before stronger capillary pull develops, enriching the first recovered fraction in selected proteins (e.g., glycoproteins or cytokines) while later fractions contain different biomarker distributions.

The fractionation module provides concentration at two levels: (i) selective enrichment of a fluid fraction relative to other fluid within the matrix; and (ii) volumetric concentration of biomarkers from a larger initial biological fluid volume, saliva, serum, GCF, or other, into a smaller recovered diagnostic fraction. Both effects rely on spatial capture, transport bias, partitioning, and controlled release rather than chemical amplification, and may together achieve concentration factors of at least 10×, 50×, 100×, 500×, 10³×, or greater. In diagnostic contexts where salivary biomarker levels are diluted relative to serum by factors of approximately 10⁻¹ to 10⁻³, this architecture may restore or exceed serum-equivalent detectability.

Elution volume and timing are governed by cartridge geometry, absorbent capacity, staged wicks, channel resistance, valves, or fixed-volume reservoirs. Wick performance may be tuned by material selection, pore size, wettability, geometry, or preconditioning. Narrow microchannels, staged wicks, or variable cross-sectional conduits regulate flow velocity and extraction force. A first wick may initiate release chemistry and permit detachment of smaller or more weakly retained proteins; a second wick with greater capillary pull may subsequently increase extraction force, promoting release of larger proteins or deeper-region species and transporting eluate toward a detection zone. Early eluate may be directed to a detection region while later eluate is passively diverted to waste, providing reproducible volumetric preconcentration across users.

Eluate flow may be fractionated into temporally or spatially distinct portions. For example, an initial ~5 uL volume may contain elevated levels of large glycosylated proteins while a subsequent ~10 uL fraction contains relatively higher levels of small signaling proteins; alternatively, capillary geometry may be configured so that early eluate contains both small and large proteins at elevated levels relative to remaining matrix fluid. Fractionation arises from differential binding strength, diffusion distance, steric accessibility, electrostatic partitioning, matrix deformation, or combinations.

Concentration factors depend upon analyte abundance, binding affinity, matrix architecture, elution kinetics, and recovery volume control. Multiplex embodiments may produce different concentration factors across analytes, particularly where capture regions target distinct biomarker classes, metabolic markers, inflammatory proteins, infectious disease markers, glycoproteins, cytokines, viral antigens, or bacterial components.

The fractionation module cooperates with the configuration module so that spatially distinct capture regions release biomarkers sequentially, surface-proximal regions into early fractions, deeper zones into later fractions, and with the transport module so that target biomarkers localize in regions accessed by early recovery flow, increasing enrichment. Dynamic mechanical loading (compression, decompression, or mastication) alters pore geometry, hydraulic permeability, and interstitial fluid flux, changing timing and composition of recovered fractions; cyclic deformation introduces non-linear strain-dependent permeability, capillary pressure variation, and matrix-solute interaction effects that modify recovery kinetics.

The fractionation module thus provides a controlled fluid extraction architecture in which a selectively recovered fraction exhibits increased biomarker concentration relative to fluid remaining in the matrix, and may also represent volumetric concentration relative to the original biological fluid sample, increasing apparent diagnostic sensitivity through spatial and fluidic design rather than chemical amplification. The fractionation module may be employed alone or with the configuration module, transport module, and signal module.

### Detailed Description of Diagnostic and Assembly Processes

The diagnostic platform incorporates diagnostic processes (computational logic) that transforms raw detection signals into clinically interpretable outputs. Signal processing, normalization, and index computation are tightly coupled to the platform's physical sampling and recovery architecture, operating on measurements that reflect the specific capture, transport, and elution conditions of each sampling event. The performance characteristics described in preceding sections depend on assembly processes (fabrication) capable of reproducing the spatial, compositional, and functional properties of the sampling subsystem. This section describes methods and material strategies used to construct the fluid-accessible matrix, transport and recovery elements, detection components, and their integration into a complete diagnostic platform.

### Computational Logic and Diagnostic Interpretation

In certain embodiments, the diagnostic platform includes computational logic configured to process signals derived from biomarkers captured, partitioned, transported, released, or detected by the sampling subsystem and associated modules. The computational logic operates on measurements from physical interactions within or adjacent to the fluid-accessible matrix, including fluid exchange, biomarker binding, staged elution, deformation cycles, and detection events. Computational logic may be implemented in hardware, software, firmware, or combinations thereof, and may execute locally in a reader or cartridge, in a handheld or mobile device, or remotely in a network-connected system.

In certain embodiments, computational logic receives signals from one or more detection modalities, including: optical signals (colorimetric intensity, fluorescence emission, reflectance, luminescence); electrical signals (current, voltage, impedance, conductance, capacitance); image-derived signals from the matrix or associated assay regions; and time-dependent signals associated with elution flow, deformation cycles, or signal kinetics. These signals correspond to concentrations or relative abundances of biomarkers captured or released by the sampling subsystem.

Preprocessing may include background subtraction, noise filtering, spatial averaging, baseline correction using reference zones, or correction using internal normalization markers from the same sampling event. Calibration parameters associated with a specific matrix configuration, ligand set, multiplex panel, or cartridge architecture may be retrieved from a stored memory element, identifier, or external database.

In certain embodiments, normalization compensates for variability in physical sampling parameters including biological fluid volume contacted, matrix hydration state, mechanical deformation cycles, sampling duration, elution volume, transport bias conditions, and recovery timing, obtained from mechanical sensors, timing data, image analysis, electrical measurements, or signal kinetics. A non-limiting normalization procedure computes:

$$Normalized Value = Signal Target / Signal Reference$$

where Signal Target is a measured biomarker signal and Signal Reference is a normalization marker captured in the same sampling event. Multivariate normalization using multiple normalization markers and/or sampling metadata (deformation count, estimated elution volume, flow characteristics) is also contemplated. Computational logic may interpret signals in view of the volume concentration achieved by the platform.

The platform contacts a volume of biological fluid (V_contacted) during sampling and delivers a smaller recovered volume (V_delivered) to the detection subsystem, with V_contacted exceeding V_delivered by at least one, and in certain embodiments at least two, orders of magnitude. The effective concentration gain (E) is:

$$E = n \times ( V\_contacted / V\_delivered )$$

where n represents capture and recovery efficiency. With V_contacted in the milliliter range and V_delivered in the microliter range, E falls in the range of 10²-10⁵ or greater. Computational logic may account for known dilution characteristics of salivary biomarkers relative to serum and interpret concentrated salivary fractions accordingly. This volumetric concentration also improves statistical detectability of low-abundance biomarkers: by accumulating greater absolute molecule numbers prior to detection, the platform reduces Poisson-limited relative noise without chemical amplification.

In multiplex embodiments, computational logic computes indices derived from combinations of normalized biomarker values, including ratios, weighted sums, regression-based transformations, threshold comparisons, or multivariate classification models. For example, a periodontal index may combine normalized metalloproteinase and inflammatory marker signals; a metabolic index may combine metabolite and protein-based markers; and infectious disease panels may incorporate antigen detection and host-response markers. Multiplex panels may target metabolic disease, liver disease, HIV, tuberculosis, inflammatory conditions, or other disease states, with differential concentration factors across analyte classes incorporated into computational weighting.

Indices may be compared to individualized baseline profiles and longitudinal trends to establish personalized thresholds and detect deviations, improving sensitivity for disease detection or therapeutic monitoring. Outputs, including classifications, trend indicators, alerts, or severity scales, may be displayed as visual indicators, transmitted to a mobile application, stored for longitudinal tracking, or communicated to a remote system. Computational logic remains coupled to the physical sampling, capture, transport, and recovery architecture and does not operate independently of the platform's physical transformations.

### Fabrication and Assembly

In certain embodiments, the diagnostic platform is fabricated with processes that establish spatial capture organization, compositional heterogeneity, migration-biasing features, and volumetric recovery control in the matrix. The sampling subsystem may be fabricated as a unitary structure or as an assembly of coordinated components including the matrix, fluid-permeable enclosure, transport elements, and detection interface structures.

The matrix may be formed from organic, inorganic, hybrid, or composite materials capable of supporting biomarker interaction and fluid migration, any material class that supports capture, partitioning, transport bias, signal interaction, or analyte recovery. Formation methods include, without limitation: casting, extrusion, templating, printing, injection molding, additive manufacturing, freeze-drying, solvent casting, phase separation, particulate templating, fiber formation or entanglement, lamination of porous sheets, compression molding, or combinations thereof.

In certain embodiments, the matrix includes spatially distinct regions with differing structural or compositional characteristics, established by sequential formation steps, local material modification, gradient processes, layered assembly, regional crosslink variation, or selective deposition of secondary or functional materials. Fabrication may produce spatial differences in porosity, charge density, hydrophilicity, ligand density, stiffness, swelling, or mechanical compliance to influence biomarker attraction, transport, binding, retention, or release. Multiple material classes may be combined to produce heterogeneous functional domains whose coupled or non-linear interactions among transport, binding, and partitioning yield emergent enrichment effects.

Immobilized capture domains may be incorporated during or after matrix formation by covalent attachment, affinity anchoring, physical adsorption, entrapment during polymerization, surface functionalization, conjugation to scaffold-bound linkers, or incorporation of functionalized particles. In certain embodiments, capture ligands are associated with particles or microstructures localized within defined matrix regions, surface-proximal zones, depth-distributed regions, radial regions, axial bands, cascaded layers, or parallel flow paths, with different ligand-bearing populations creating multiplex capture territories. Structures configured to influence biomarker migration, including transport-biasing features that establish gradients or directional transport through spatial material variation rather than external fluid handling, may be formed intrinsically within the matrix or assembled as discrete elements in fluid communication with it.

Structured flow-control elements, including absorbent structures, microstructured conduits, staged capillary elements, defined flow-resistance geometries, or fixed-volume or metered reservoirs, may regulate recovery volume and fractionation behavior. Such elements may be fabricated from cellulose, polymeric membranes, sintered materials, microfabricated polymers, or molded thermoplastics to produce selective, reproducible volumetric concentration without user handling.

Signal-generating elements incorporated during assembly may include conductive or optical elements, transparent or sensing regions within the enclosure, reactive or reporting elements in fluid communication with recovered fractions, or electrical coupling to a reader device. Detection elements, including electrochemical electrodes, conductive traces, optical windows, photodetectors, colorimetric or fluorescence assay regions, impedance sensing elements, or wireless antenna structures, may be co-fabricated with the matrix or assembled into a cartridge or housing. The fluid-permeable enclosure may be fabricated from polymeric materials, elastomers, meshes, membranes, or perforated shells, with permeability, pore size, and thickness selected to regulate fluid entry and provide mechanical protection.

Assembled sampling systems may be sterilized using gamma irradiation, ethylene oxide, electron beam treatment, or other methods compatible with ligand stability and matrix integrity, and packaged in sealed pouches, cartridges, or multi-unit assemblies to preserve hydration state or dryness as required. Fabrication and assembly processes are selected to maintain alignment among the matrix capture architecture, transport bias, recovery metering, and signal readout structures, integrating sampling, transport, recovery, signal-generation, and computational logic into a single functional system.

### Detailed Description of Embodiments

### Introduction: Shared Implementation Definitions

In the following embodiments, certain processes and structures recur across multiple embodiments and involve combinations of multiple elements or steps.

To avoid needless repetition, these are first defined independent of any particular embodiment, and their subject headings, italicized, are subsequently used in place of repeated full descriptions. The modules associated with a shared implementation definition are noted.

### Volumetric Sampling and Concentration Process:

In this process, the platform comprises a matrix retained by a fluid-permeable enclosure configured for oral use. Saliva may enter the matrix through one or more fluid-permeable regions of the enclosure, and the matrix contacts a larger oral fluid volume over a sampling interval while yielding a smaller recovered fraction enriched in one or more target biomarkers. The platform functions as a volumetric molecular integrator that converts dilute biomarker content in saliva, or in saliva mixed with GCF, into a smaller diagnostically useful fraction. In embodiments, the matrix comprises one or more hydrogel, cellulose, silica, collagen, silk, polyvinyl or hydrocolloid-based material, or composite material regions, and may include a heterogeneous architecture with two or more material phases. [configuration module, transport module, signal module, fractionation module]

### Conditioned Entry and Biased Routing Arrangement:

In this arrangement, the transport module includes one or more charged regions, pore-size gradients, capillary-accessible geometries, staged-permeability regions, wicking elements, fluidic conduits, or combinations positioned within, adjacent to, or upstream of the matrix. An upstream conditioning region may reduces mucin fouling, particulate interference, or nonspecific adsorption before saliva reaches a downstream biomarker capture region. The upstream conditioning region may comprise a non-hydrogel porous cellulose-or silica-based region, while one or more downstream antibody, antigen, or nucleic-acid capture regions comprise a hydrogel region. In embodiments an electrostatically biased region increases local availability of nucleic acids or negatively charged biomolecular complexes to a nucleic-acid capture region. One or more enclosure regions may differ in porosity, wettability, thickness, charge, or permeability so that saliva entering a first enclosure region accesses an antibody-focused capture zone, while saliva entering through a second enclosure region accesses an antigen-focused or nucleic-acid-focused capture zone. [transport module]

Nucleic-acid-biased Localization Arrangement:

In this arrangement, the nucleic-acid capture region comprises one or more charged polymer domains, ion-exchange groups, cationic microdomains, or electrostatically interactive regions that bias negatively charged nucleic acids or nucleic-acid-containing particles toward the nucleic-acid capture region. The nucleic-acid capture region may be positioned deeper within the matrix than one or more antibody-capture or antigen-capture regions, so that different analyte classes are localized according to size, diffusivity, charge, or binding kinetics. [configuration module, transport module]

### Reversible Retention and Release Processes:

In these processes, biomarkers captured within the matrix are retained reversibly and subsequently released to generate an enriched eluate. In embodiments, release occurs by change in pH, in ionic strength, change in hydration state, capillary-driven dehydration, aptamer displacement, competitive displacement, oligonucleotide displacement, competitive elution, cleavage of a cleavable tether, cleavable moieties, chaotropic exposure, thermal modulation, or their combinations. A shallow surface-proximal capture architecture may cooperate with the release conditions so that released biomarkers are removed before substantial rebinding occurs. [configuration module, fractionation module]

### Fractionated Recovery Arrangement:

In this arrangement, the platform includes a fractionated recovery arrangement under which a defined portion of fluid is recovered from the matrix, and the recovered portion is enriched in one or more biomarkers relative to fluid remaining within the matrix or recovered later. In embodiments, capillary channels, porous wicks, staged porous conduits, microfluidic pathways, reservoirs, valves, flow-resistance features, or combinations are arranged so that an initial recovered fraction is directed toward a detection region while later-recovered fluid is diverted, withheld, or directed to waste. In embodiments, the first several microliters of recovered eluate contain a higher relative concentration of targeted biomarkers than later-recovered fractions. In embodiments, early-fraction enrichment arises from spatial capture architecture, surface-proximal localization, pathway-aligned release, staged hydration change, staged capillary pull, or combinations. [fractionation module]

### Post-capture Signal-development Arrangement:

In this arrangement, labeled detection antibodies, aptamers, enzymes, fluorophores, nanoparticles, or combinations are introduced in the matrix after biomarker binding, enabling sandwich-complex formation and signal generation without transfer to a separate diagnostic device. A sealed compartment or cartridge, with an optical window and one or more reagent reservoirs, may deliver labeled ligands into the matrix upon closure, enabling user-readable fluorescence or color development without user pipetting and without exposing biological fluid. The matrix may be placed in functional association with one or more electrodes so that binding of labeled detection ligands produces a measurable current or impedance change corresponding to one or more biomarkers. [signal module]

### Cartridge-coupled Recovery Processes:

In these processes, a cartridge coupled to the sampling assembly contains a metered elution blister pack, fixed-volume reservoir, absorbent pad, wick, porous conduit, microchannel, valve, flow-resistance feature, or combinations. Closure of the cartridge may initiate release and recovery by piercing the blister pack and directing a controlled volume of liquid through or across the matrix. In some embodiments, an initial recovered fraction of about 5-20 uL is directed toward a detection region, while later-recovered fluid is directed to waste, withheld, or bypassed. [fractionation module]

### Integrated Signal-generation Assemblies:

In these assemblies, the platform comprises one or more signal-producing elements positioned at the sampling assembly, within the matrix, at the enclosure, or in a detection subsystem fluidically coupled to the matrix. The signal-producing elements may comprise labeled detection ligands, enzymes, fluorophores, nanoparticles, electrodes, optical reporter regions, impedance elements, lateral-flow strips, nucleic-acid amplification regions, sequencing workflows, PCR-based workflows, or combinations. Detection may be colorimetric, fluorescent, electrochemical, optical, impedance-based, or otherwise measurable. [signal module]

### Computational Logic:

Computational logic interprets one or more generated signals using sampling duration, recovered volume, concentration factor, analyte ratio, hydration state, matrix deformation cycles, baseline comparison, normalization relative to sample volume, elapsed sampling time, internal reference markers, prior measurements from the same subject, longitudinal trends, population-based profiles, external reference standards, stored threshold values, or combinations. In embodiments, the computational logic converts these signals into a classification output, normalized values, analyte ratios, composite indices, alerts, exposure classification, infection-monitoring outputs, severity indication, trend indication, risk indicators, treatment-response outputs, progression assessments, combinations, recommendations for confirmatory testing, or other diagnostic interpretation.

### Representation of Diagnostic Platform

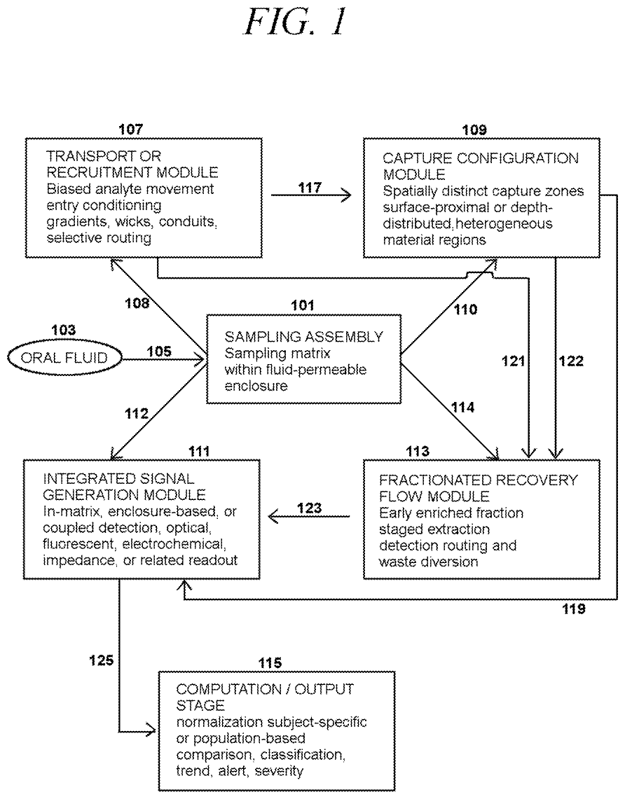

A schematic overview of a diagnostic platform is provided in FIG. 1, showing both independent and combined use of the modules for capture-configuration 109, transport 107, integrated signal-generation 111, and fractionated recovery-flow 113, in relation to a fluid-accessible matrix, with optional computational interpretation and output 115. The platform includes a sampling assembly 101 that includes a matrix within a fluid-permeable enclosure, and the one or more modules 107, 109, 111, 113 that may be used independently or in combination within different embodiments. Sample contact involves the oral fluid 103 moving 105 to the sampling assembly 101. Fluid 103 enters the matrix through the fluid-permiable enclosure. Transport or recruitment module 107 involves one or more features that condition, direct, partition, or concentrate selected elements. Capture-configuration module 109 involves one or more spatially differentiated capture regions that retain selected analytes. Integrated signal generation module 111 involves one or more signals that are generated in the matrix, at the enclosure, or in a coupled detector. Fractionated recovery flow module 113 involves directing a specific recovered fraction to detection, while later fluid is withheld, bypassed, or sent to waste. The computation/output stage 115 processes, interprets, and communicates signal data.

Sampling assembly 101 sends flows to each module, flow 108 to transport module 107, flow 110 to configuration module 109, flow 112 to signal generation module 111, and flow 114 to fractionated recovery flow module 113. Transport module 107 output may flow 117 to configuration module 109. Configuration module 109 output may flow 119 to signal generation module 111. Transport module 107 output may flow 121 to fractionated recovery module 113. Configuration module 109 output may flow 122 to fractionated recovery module 113. Fractionated recovery module 113 output may flow 123 to integrated signal generation module 111. Signal generation module 111 output may flow 125 to the computation/output stage 115. Any feed may occur without all the others.

### Embodiments

### 1. Xerostomia

Xerostomia is a core embodiment that also provides a reusable shared implementation definition, the active fluid-exchange and GCF-mobilization process for fluid exchange with the oral environment, including GCF liberation and reabsorption.

Saliva contains a wide range of biomarkers that reflect oral and systemic disease states, including proteins, antibodies, nucleic acids, metabolites, extracellular vesicles, and microorganisms. Although salivary diagnostics offer clear advantages as a non-invasive alternative to blood sampling, their practical utility may be limited for individuals with reduced salivary flow or xerostomia, which are common in populations affected by autoimmune disease, neurodegenerative disease, cancer therapy, medication use, aging, or chronic stress. Existing saliva collection methods rely on passive drool, swabs, or sponges and frequently fail to obtain sufficient sample or retain low-abundance biomarkers under such conditions.

The diagnostic platform may be configured for use in subjects with reduced salivary flow, including xerostomia, or for other conditions where inadequate saliva or GCF is sampled. The matrix, in the active fluid-exchange and GCF-mobilization process, actively exchanges fluid with the oral environment by donating fluid into the oral cavity during sampling, mixing with saliva and liberating GCF, then reabsorbing the mixed fluid, thereby improving analyte capture even when salivary flow is limited. This process is not limited to xerostomia and may also be used in other embodiments involving low-flow sampling conditions, GCF-borne analytes, or biomarkers that benefit from enhanced whole-mouth fluid exchange. This approach protects fragile analytes from degradation and transforms saliva from a dilute and variable fluid into a reliable diagnostic sample suitable for non-invasive screening, monitoring, and research across a wide range of disease contexts.

### 2. Tuberculosis

In certain embodiments, the diagnostic platform is configured to capture, concentrate, recover, and optionally detect salivary biomarkers associated with tuberculosis. Such biomarkers may include Neural Cell Adhesion Molecule (NCAM), Neural Cell Adhesion Molecule (SAP), Fractalkine, IL-1B, IL-13, IL-1a, Granulocyte-Macrophage Colony-Stimulating Factor (GM-CSF), or their combinations. The platform may be configured for multiplex detection of two, three, four, or more tuberculosis-associated biomarkers within a single sampling event.

In certain embodiments, the platform comprises a fluid-accessible matrix retained by a fluid-permeable enclosure and configured for oral use. A volumetric sampling and concentration process yields a recovered fraction enriched in one or more tuberculosis-associated biomarkers. A biased movement configuration may include a reduced-fouling entry layer, a pore gradient, a crosslink gradient, or a wick-assisted entry path. These features can improve cytokine access and reduce mucin masking.

In certain embodiments, a conditioned entry and biased routing arrangement may be used to reversibly retain tuberculosis-associated biomarkers. The platform includes a transport module configured to bias movement of saliva or selected biomarker classes toward one or more tuberculosis-related capture regions.

In certain embodiments, the platform includes a capture-configuration module comprising two or more spatially distinct capture regions arranged within the matrix. A first capture region may be configured to capture one or more cytokines or chemokines including IL-1B, IL-13, Fractalkine, IL-1a, or GM-CSF, and a second capture region is configured to capture one or more larger proteins such as NCAM or SAP. The cytokine-focused capture region may have a higher ligand density than the larger-protein capture region, to increase encounter probability for low-abundance analytes. One or more protein-focused capture regions may be positioned within about 50-300 um of a fluid-entry surface of the matrix, and a shallow lower-crosslink-density region may improve access to NCAM epitopes. Affinity ligands may include antibodies, antibody fragments, aptamers, peptides, receptor-mimetic ligands, or their combinations. Antibodies may be oriented by Protein A/G, site-directed coupling, or other orientation-preserving attachment chemistry, reducing strain and improving fit, to improve effective association.

In certain embodiments, a tuberculosis four-plex comprises a first capture region configured for NCAM, a second capture region configured for SAP, a third capture region configured for IL-1B, and a fourth capture region configured for IL-13. In other embodiments, NCAM and SAP are co-localized in a protein zone and IL-1B and IL-13 are co-localized in a cytokine zone beneath a common antifouling or conditioning region. One or more capture regions may be arranged as surface-proximal zones, radial layers, parallel lanes, segregated domains, or cascaded zones.

In certain embodiments, fractalkine is captured as a soluble salivary chemokine or chemokine-domain-containing fragment. The fractalkine capture region may be positioned near a surface of the matrix beneath an antifouling or conditioning region, and comprises antibodies, antibody fragments, aptamers, or combinations directed to a fractalkine-containing target. Aptamers may be tethered by PEG spacers or other flexible linkers, to support fractalkine accessibility, reduce steric hindrance, and create more favorable aptamer presentation, factors important for that analyte. The fractalkine capture region may be combined with a neighboring or overlapping capture region configured for IL-1a, GM-CSF, or another tuberculosis-associated biomarker.

In certain embodiments, the platform further includes a fractionated recovery-flow module configured as a fractionated recovery arrangement to selectively recover a defined portion of fluid from the matrix, wherein the recovered portion is enriched in one or more tuberculosis-associated biomarkers relative to other fluid remaining within the matrix or subsequently recovered. In certain embodiments, reversible retention and release processes generate an enriched eluate. Embodiments employ cartridge-coupled recovery processes to obtain a biomarker enriched fraction in a detection region.

In certain embodiments, capillary resistance is regulated to create staged extraction behavior. Narrow microchannels, staged wicks, variable cross-sectional conduits, wettability-tuned materials, or preconditioned absorbent elements may regulate flow velocity and extraction force. A first wick may initiate release chemistry to permit detachment of one or more cytokines or chemokines, while a second wick having greater capillary pull may subsequently promote release of larger proteins including NCAM and transport of eluate toward detection. An initial fraction may be relatively enriched in NCAM and SAP, while a later fraction may contain relatively higher cytokine levels. Flow geometry may be configured so that an early recovered fraction contains elevated levels of both larger proteins and smaller cytokines.

In certain embodiments, the platform includes integrated signal-generation assemblies for detection. Labeled detection antibodies, labeled aptamers, enzymes, fluorophores, nanoparticles, or combinations thereof may be introduced into contact with the matrix after biomarker binding. A post-capture signal-development arrangement may be used, to enable sandwich-complex formation and signal generation corresponding to fractalkine, IL-1a, IL-1B, IL-13, NCAM, SAP, or combinations, without transfer to a separate diagnostic device.

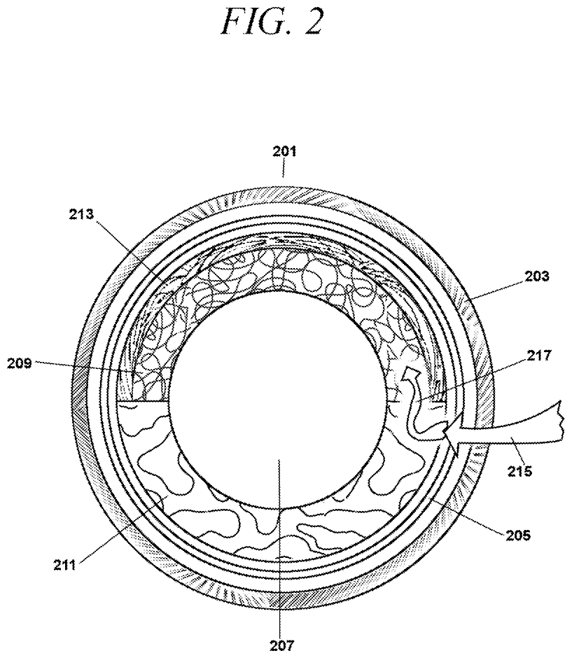

FIG. 2 illustrates a sectional functional schematic of a tuberculosis biomarker embodiment. The figure shows a sampling assembly 201 comprising a fluid-permeable enclosure 203, an outer conditioning layer 205, and a matrix 207, with spatially differentiated capture regions including a cytokine or chemokine capture region 209, a protein capture region 211, and an surface-proximal fractalkine capture region 213. In this example, saliva 215 enters through the enclosure 203 and passes through the conditioning layer 205 and a biased routing arrangement 217 orients it to the capture region 209.

In certain embodiments, the platform includes computational logic configured to interpret one or more tuberculosis-related biomarker signals. Normalized values derived from NCAM, SAP, fractalkine, IL-1B, IL-13, IL-1a, or GM-CSF, may be combined into a multiplex tuberculosis classification output, severity indication, trend indicator, or other diagnostic interpretation

Certain tuberculosis embodiments are useful for screening, triage, monitoring, serial surveillance, home collection, point-of-care evaluation, pediatric sampling, or testing of subjects unable or unlikely to provide adequate sputum volume. The platform may enable multiplex oral-fluid capture, selective enrichment, fractionated recovery, and optional integrated detection of tuberculosis-associated biomarker panels using a single sampling events.

### 3. Hepatitis A, Hepatitis B, and Hepatitis C

In certain embodiments, the diagnostic platform is configured to capture, concentrate, and optionally detect salivary biomarkers associated with hepatitis A virus (HAV), hepatitis B virus (HBV), and hepatitis C virus (HCV). Salivary biomarkers relevant to these embodiments may include one or more virus-specific antibodies, one or more viral antigens, one or more viral nucleic acids, or their combinations. The antibodies may comprise one or more of IgA, IgG, and IgM antibodies specific to HAV, HBV, or HCV antigens. The viral antigens may comprise hepatitis B surface antigen (HBsAg), hepatitis B core-associated antigenic material, hepatitis C core antigen, or their combinations. The viral nucleic acids may comprise HBV DNA, HAV RNA, HCV RNA, or fragments, amplification products, or their vesicle-associated forms.

In certain embodiments, the platform comprises a volumetric sampling and concentration process. The diagnostic platform may include a capture-configuration module comprising two or more spatially distinct capture regions arranged within the matrix. A first capture region may be configured to enrich one or more immunoglobulin classes from saliva. This region may comprise isotype-selective anti-human IgA, anti-human IgG, anti-human IgM, Fc-binding proteins, Fc-binding fragments, or their combinations. The first capture region may be positioned upstream of one or more virus-specific antibody capture regions, so that salivary immunoglobulins are first enriched, then subsequently they are exposed to immobilized viral antigens or antigenic fragments. The virus-specific antibody capture regions may comprise immobilized HAV antigens, HBV core antigens or antigenic fragments, HBsAg-derived antigens or epitopes, HCV antigens, HCV core-derived antigens or epitopes, or their combinations, selectively retaining antibodies associated with hepatitis infection or prior exposure. A first antibody-capture region may be configured for anti-HAV antibodies, a second antibody-capture region may be configured for anti-HBc antibodies, and a third antibody-capture region may be configured for anti-HCV antibodies. In other embodiments, two or more such functions are combined in overlapping, layered, or co-localized regions.

In certain embodiments, the matrix further comprises one or more antigen-capture regions configured to directly retain soluble viral antigens present in saliva. These antigen-capture regions may comprise immobilized antibodies, antibody fragments, aptamers, peptides, or other affinity ligands specific for HBsAg, HCV core antigen, or other hepatitis-associated viral proteins. One or more antigen-capture regions may be positioned as surface-proximal zones so that larger protein analytes or lower-abundance antigens are intercepted early during fluid entry. In other embodiments, one or more antigen-capture regions are depth-distributed or arranged in parallel pathways relative to antibody-capture regions. The antigen-capture regions may be chemically and spatially distinct from the antibody-capture regions, so that direct viral-antigen capture and virus-specific antibody capture occur under different fouling, transport, or release conditions.

In certain embodiments, the matrix comprises a nucleic-acid capture region configured to bind one or more hepatitis viral nucleic acids. The nucleic-acid capture region may comprise immobilized oligonucleotide probes, peptide nucleic acids, locked nucleic acids, modified nucleic acids, or other sequence-selective affinity ligands configured to hybridize with HBV DNA, HAV RNA, HCV RNA, or their conserved sequences. The nucleic-acid capture region may further comprise a nucleic-acid-biased localization arrangement. In this respect, the matrix may comprise distinct capture territories for antibody biomarkers, soluble antigen biomarkers, and nucleic-acid biomarkers within a single fluid-accessible matrix body.

Certain embodiments are implemented as layered sampling matrices comprising: (a) an upstream conditioning region to reduce salivary fouling or alter ionic environment; (b) an immunoglobulin-enrichment region configured to preferentially retain one or more of IgA, IgG, and IgM; (c) one or more virus-specific antibody capture regions presenting HAV, HBV, or HCV antigens or antigenic fragments; (d) one or more direct viral-antigen capture regions configured to bind HBsAg, HCV core antigen, or both; and (e) a nucleic-acid capture region configured to bind HBV DNA, HAV RNA, HCV RNA, or their combinations. The matrix may be arranged in parallel lanes, radial layers, concentric annuli, shell-and-core regions, or patterned subdomains, each configured to preferentially localize a different hepatitis biomarker class.

In certain embodiments, the diagnostic platform further includes a transport module configured to bias movement of saliva or selected biomarker classes toward one or more hepatitis-related capture regions. The transport module comprises the conditioned entry and biased routing arrangement, with one or more hepatitis-associated biomarkers as targets.

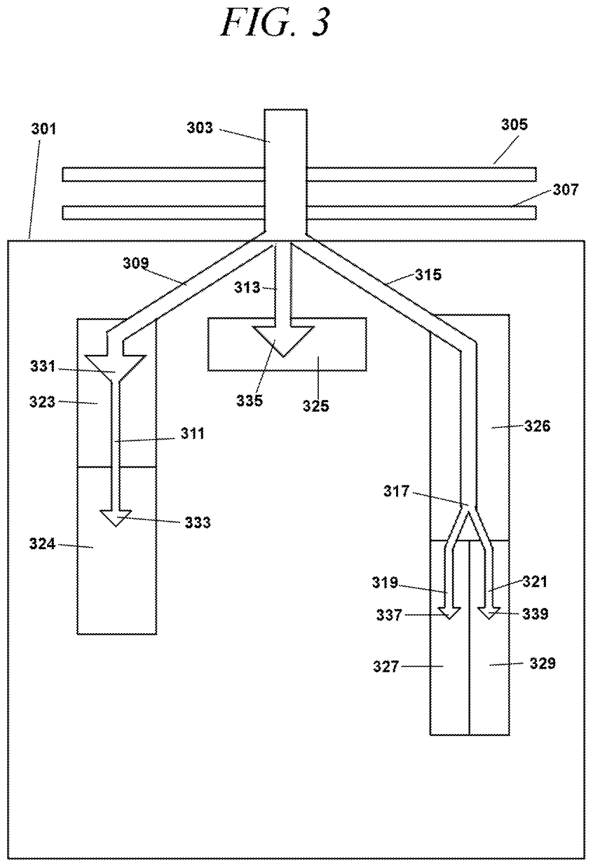

FIG. 3 illustrates a hepatitis embodiment in which a matrix 301 retained by a fluid-permeable enclosure 305 includes distinct internal capture territories for antibodies, viral antigens, and viral nucleic acids. Saliva stream 303 passes through the fluid-permeable enclosure 305 and an upstream conditioning region 307, the saliva stream 303 partitioned into parallel or laterally separated saliva pathways including an immunoglobulin/virus specific saliva pathway 309, and a part of saliva from this pathway becomes a virus-specific antibody saliva pathway 311; a surface-proximal direct antigen saliva pathway 313, and a nucleic-acid saliva pathway 315 that bifurcates 317 into a charge-biased localization saliva pathway 319 and an RNA/DNA saliva pathway 321. The matrix 301 includes an immunoglobulin enrichment region 323 over a virus-specific antibody capture region 324, a separate surface-proximal viral-antigen capture region 325, and separate region with a surface-proximal area that facilitates nucleic-acid movement 326, surface deeper charge-biased localization region 327 and nucleic-acid capture region 329. Part of the immunoglobulin/virus saliva pathway 309 is sequestered 331 in the immunoglobulin enrichment region 321, and part of it continues as the virus-specific antibody saliva pathway 311 and is sequestered 333 in the virus-specific antibody capture region 324. The surface-proximal direct antigen-capture saliva pathway 313 is sequestered 335 in the surface-proximal viral-antigen capture region 325. The charge-biased localization saliva pathway 319 is sequestered 337 in the charge-biased localization region 327, and the nucleic-acid capture saliva pathway 319 is sequestered 339 in the nucleic-acid capture region 329.

In certain embodiments, biomarkers captured within the matrix are retained reversibly and subsequently released to generate a diagnostically useful eluate, through reversible retention and release processes.

In certain embodiments, the diagnostic platform further includes a fractionated recovery flow module configured to selectively recover a defined portion of fluid from the matrix, wherein the recovered portion is enriched in one or more hepatitis biomarkers relative to other fluid remaining within the matrix or subsequently recovered. A fractionated recovery arrangement may be employed, to obtain recovered eluate with a higher relative concentration of hepatitis-related antibodies, viral antigens, viral nucleic acids, or their combinations than other recovered fractions.

In certain embodiments, the platform further includes a signal module. The integrated signal-generation assemblies may be employed, to generate a signal corresponding to one or more hepatitis biomarkers captured in the matrix or recovered from the matrix. Computational logic may interpret one or more generated signals and improve interpretation of salivary hepatitis biomarker profiles.

In certain embodiments, the disclosed hepatitis platform is useful for screening, triage, monitoring, surveillance, or home or point-of-care collection. The platform may be configured for multiplex use so that a single oral sampling event yields information regarding one or more antibody markers, one or more direct viral antigens, and one or more viral nucleic-acid markers. In this manner, the matrix provides not merely passive saliva absorption, but a spatially organized capture, concentration, transport, recovery, and optional detection architecture for salivary hepatitis biomarker analysis.

### 4. Liver Disease

In certain embodiments, the diagnostic platform is configured to capture, concentrate, recover, and optionally detect salivary biomarkers associated with liver disease, including liver fibrosis, cirrhosis, and related chronic hepatic conditions. The biomarkers may include hyaluronic acid (HA), tissue inhibitor of metalloproteinase-1 (TIMP-1), a-2-macroglobulin (a2M), type III procollagen peptide, y-glutamyl transferase, total bilirubin, aspartate aminotransferase, alanine aminotransferase, or combinations. The platform may be configured for multiplex capture of two, three, four, or more liver-associated biomarkers within a single sampling event.

In certain embodiments, the diagnostic platform comprises a super-absorbent hydrogel preloaded with at least 95-99% water. An active fluid-exchange and GCF-mobilization process increases availability of GCF-borne analytes for capture by matrix affinity domains.

In certain embodiments, liver biomarker detection is enabled with the volumetric sampling and concentration process that enriches liver-associated biomarkers from saliva, saliva mixed with GCF, or both. The matrix may include a heterogeneous architecture with different material phases selected for polysaccharide capture, glycoprotein capture, GCF mobilization, or combinations.

In certain embodiments, liver biomarker detection is enabled with the conditioned entry and biased routing arrangement, with one or more downstream HA, TIMP-1, or a2M capture regions comprising hydrogel regions. Biased routing may include cationic zones (PLL, chitosan, quaternary amines) that attract HA electrostatically, leading to differential entry of bulk saliva versus GCF-associated species, or preferential localization of transport-limited proteins near the matrix surface, increasing local HA concentration for downstream receptors.

In certain embodiments, the platform includes a capture-configuration module with spatially distinct capture regions arranged to localize liver biomarkers according to size, charge, diffusivity, source fluid, binding chemistry, or release behavior. A first capture region may be configured for HA, a second capture region may be configured for TIMP-1, and a third capture region may be configured for a2M. The regions may be co-located, layered, cascaded, parallel, or hybrid. Larger or more transport-limited biomarkers may be captured closer to the matrix surface, while smaller or more readily diffusing biomarkers penetrate farther before binding.

In certain embodiments, HA is captured in a surface-accessible region configured for a large, strongly anionic polysaccharide. The HA region may comprise HABP ligands tethered by PEG spacers beneath an antifouling skin in a low-crosslink surface layer. The HA region instead may comprise phenylboronic acid groups configured to bind cis-diols. The HA region may include CD44 ectodomains, CD44-derived peptides, or multivalent CD44 assemblies to increase avidity.

In certain embodiments, TIMP-1 is captured in a shallow protein-access region beneath an antifouling surface layer. The TIMP-1 region may comprise anti-TIMP-1 antibodies immobilized by Protein A/G, maleimide coupling, EDC/NHS coupling, engineered tags, or combinations. The TIMP-1 region may comprise aptamers tethered by PEG spacers, molecularly imprinted domains based on a TIMP-1 peptide sequence, or combinations. The TIMP-1 region may have large pores and lower crosslink density so that TIMP-1 glycoproteins can access ligands with reduced steric interference. One or more prefilter, mucin-depletion, size-exclusion, or sacrificial surface features may reduce background interference before TIMP-1 reaches the capture region.

In certain embodiments, a2M is captured in a shallow high-access region configured for a very large GCF-associated glycoprotein. The a2M region may lie immediately beneath a reduced-fouling surface layer and comprise immobilized anti-a2M antibodies or related affinity ligands. The surrounding matrix may be super-absorbent and highly water-loaded so that, during oral use, it solubilizes and mobilizes GCF from gingival margins and sulcular regions, thereby increasing availability of a2M and other GCF-borne analytes. Hydrophilic domains, ionic polymer domains, capillary exchange, interfacial fluid exchange, osmotic gradients, or combinations may increase transport of solubilized GCF into the matrix before selective capture. Protease inhibitors, EDTA, or both may be present to reduce a2M degradation or activation during sampling.

In certain embodiments, different liver biomarkers are captured with different affinity chemistries within the same matrix. HA may be captured by HABP, PBA, CD44-derived capture systems, or combinations. TIMP-1 may be captured by antibodies, aptamers, peptide-imprinted domains, or combinations. a2M may be captured by antibody-based affinity systems or related selective binders. A first capture mechanism may recruit or preconcentrate one or more analytes, and a subsequent capture mechanism provides molecular specificity. Such mechanisms may be arranged in a cascade so that capture depends on both transport bias and ligand selectivity.

Certain embodiments use the reversible retention and release processes. HA may be released by lowering pH, adding sorbitol, or other boronate-disrupting conditions when PBA-based capture is used. TIMP-1 may be released by low-pH glycine exposure followed by neutralization, by competitive elution with an epitope peptide or binder mimetic, by salt or pH shift in aptamer-based systems, by complementary oligonucleotide displacement, or by other release conditions. a2M may be released using antibody-elution systems described herein, including pH shift, competitive elution, cleavable tether release, or combinations.

Certain embodiments use the fractionated recovery arrangement, such that an initial enriched fraction is directed toward detection while later-recovered fluid is diverted, withheld, or directed to waste. The fractionation profile may be selected so that shallow HA, TIMP-1, or a2M regions contribute disproportionately to early recovered fractions. All captured analytes may be eluted into a common recovery volume, or sequential elution may be used so that one class of liver biomarkers is released before another, thereby enabling independent quantification of HA, TIMP-1, a2M, or additional fibrosis-related analytes.

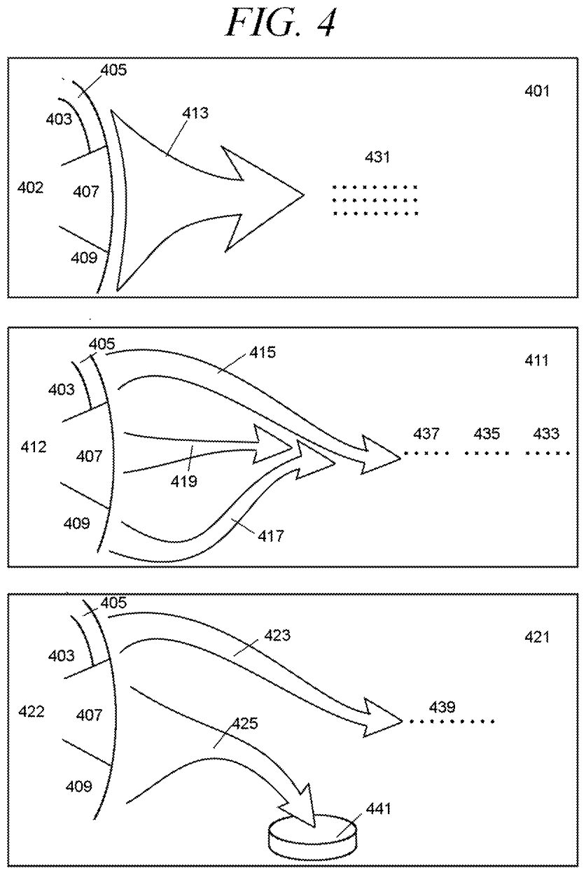

FIG. 4 illustrates a fractionated recovery arrangement for a multiplex liver disease embodiment. The figure shows three sampling apparatus 401, 411, 421, each configured with a matrix, all the matrices having the same architecture. Each matrix has distinct liver-biomarker capture territories including an HA region 403 beneath cationic domains 405 that increase HA concentration, a TIMP-1 region 407, and an a2M region 409. HA may be released under boronate-disrupting conditions including pH reduction or sorbitol addition, TIMP-1 may be released by low-pH glycine exposure, competitive elution, salt or pH shift, complementary oligonucleotide displacement, or combinations, and a2M may be released using antibody-elution systems as previously described. Sampling apparatus 401 matrix 402 has all captured analytes 413 released as a common recovery volume. Sampling apparatus 411 has a sequential recovery arrangement, with different release conditions causing a first analyte class 415 to be released before a second analyte class 417, which is released before a third analyte class 419. Sampling apparatus 421 has an initial fraction 423 that goes to detection, while later fluid 425 goes to waste or bypass 441. The different paths lead to different readouts. Sampling apparatus 401 has a common multiplex readout 431 that simultaneously analyzes HA, TIMP-1 and a2M. Sampling apparatus 411 has sequential readouts for independent quantification of a2M 437, TIMP-1 435, and HA 433. Sampling apparatus 421 has an early fraction 423 detected 439, while the later fraction 425 goes to waste 441.

In certain embodiments, the platform includes integrated signal-generation assemblies. In embodiments, a first signal corresponds to HA, a second signal corresponds to TIMP-1, and a third signal corresponds to a2M. Signal-producing elements may be arranged in region-specific fashion so that different liver biomarkers produce distinguishable outputs during common or sequential recovery.

Certain embodiments use computational logic to interpret one or more generated signals from a single biomarker or from a multiplex fibrosis panel. Normalized values, analyte ratios, weighted combinations, or composite fibrosis indices may be calculated from HA, TIMP-1, a2M, or additional liver-associated analytes. The computational interpretation may account for differential concentration behavior across analyte classes, including polysaccharides, glycoproteins, and GCF-enriched proteins.

Certain embodiments are useful for screening, triage, fibrosis staging, cirrhosis monitoring, longitudinal surveillance, low-cost community testing, or identification of patients who should receive further evaluation or intervention. Multiplex capture, volumetric concentration, selective routing, and sequential or common recovery may improve sensitivity and flexibility for salivary assessment of liver disease in settings ranging from high-precision specialty workflows to low-cost field deployment.

### 5. Cardiovascular Disease

In certain embodiments, the diagnostic platform is configured to capture, concentrate, recover, and optionally detect salivary biomarkers associated with cardiovascular disease, including coronary artery disease, acute cardiac injury, heart failure, or related cardiac conditions. The biomarkers may include small extracellular vesicles (sEVs), vesicle-associated proteins, intravesicular cardiac biomarkers, vesicle-derived nucleic acids, or combinations. The cardiac biomarkers may include creatine kinase myocardial band (CK-MB), cardiac troponin I, cardiac troponin T, myoglobin, C-reactive protein (CRP), Cystatin C, Cystatin S, B-type natriuretic peptide (BNP), N-terminal pro-BNP (NT-proBNP), high-sensitivity CRP (hs-CRP), or combinations. In embodiments, a volumetric sampling and concentration process concentrates such biomarkers from saliva, improving sensitivity and reliability of non-invasive cardiac screening, risk stratification, prognosis, treatment-response monitoring, or longitudinal disease monitoring.

Certain embodiments use the active fluid-exchange and GCF-mobilization process to enrich intact sEVs and vesicle-associated biomarkers obtained from saliva, saliva mixed with GCF, or both. The matrix may comprise one or more hydrogel, polyvinyl alcohol, cellulose-based, silica-based, hydrocolloid-based, or composite regions. The matrix may include a heterogeneous architecture in which a first region preferentially interacts with intact vesicles and a second region preferentially receives, binds, or processes vesicle-derived internal biomarkers.

In certain embodiments, the platform includes a capture-configuration module with spatially or functionally distinct capture regions arranged to perform staged handling of vesicles and vesicle-derived contents. A first capture region may comprise a first set of capture ligands specific for sEV surface markers, and a second capture region may comprise a second set of capture ligands specific for biomarkers contained within sEVs. The first set of capture ligands may bind tetraspanins including CD9, CD63, or CD81, integrins, adhesion molecules, immune-associated proteins, disease-associated surface antigens, lipid components, glycoproteins, or combinations. The second set of capture ligands may bind intravesicular cardiac proteins including CK-MB, troponin I, troponin T, myoglobin, CRP, Cystatin C, Cystatin S, BNP, NT-proBNP, hs-CRP, or combinations. In embodiments, the second set additionally or alternatively includes nucleic-acid capture probes for RNA or DNA released from sEVs.

In certain embodiments, the first capture region retains intact sEVs during fluid exchange, while a downstream or adjacent second capture region is configured to receive vesicle contents after permeabilization or lysis. The first and second capture regions may be arranged as surface-proximal and deeper regions, adjacent parallel lanes, cascaded zones, or other spatially differentiated territories. This arrangement may separate vesicle-surface recognition from intravesicular biomarker capture so that intact vesicles and released internal contents need not be processed at the same location. In embodiments, one or more cardiac biomarkers associated with the vesicle exterior may be captured without prior vesicle permeabilization.

In certain embodiments, the first capture region includes antibodies or antibody fragments oriented to preserve vesicle-binding activity. Orientation may be achieved by site-specific conjugation through the Fc region, hinge region, engineered cysteine residues, Protein A/G, biotin-streptavidin linkages, or combinations. In embodiments, the first capture region instead or additionally includes aptamers, peptides, affinity ligands, lipid-interacting moieties, lectins, sulfated glycosaminoglycans, heparin, ion-exchange groups, charged polymer domains, or combinations configured to bind sEV surface proteins, lipids, glycoproteins, vesicle-protein coronas, or surface charge characteristics. The first capture region may comprise multiple binding agents arranged uniformly, spatially, or functionally to enrich different sEV subpopulations.

Certain embodiments use the conditioned entry and biased routing arrangement before saliva reaches a primary vesicle-capture region. A first transport-biasing region may recruit or localize intact vesicles by charge, affinity, capillary transport, or surface interaction, and a second region may receive vesicle-derived internal biomarkers after permeabilization. Larger or more transport-limited vesicles may be preferentially localized at or near a surface-proximal region, whereas smaller released internal biomarkers penetrate more deeply before being presented to one or more downstream capture regions.

Certain embodiments permeabilize, lyse, expose, or otherwise process captured sEVs to make internal biomarker contents available for secondary capture or downstream recovery. Permeabilization may be achieved by ionic or osmotic modulation, addition of detergents or surfactants including digitonin, thermal cycling, sonication, enzymatic digestion of the vesicle membrane, incorporation of lytic agents within the matrix that are activated by a stimulus, or combinations. Permeabilization conditions may be selected to disrupt vesicle membranes while preserving integrity of cardiac proteins, nucleic acids, or other released biomarkers. The staged sequence may comprise intact sEV capture followed by vesicle permeabilization and subsequent internal-cargo capture within the same matrix.

Certain embodiments use reversible retention and release processes to release intact sEVs from the first capture region and release internal cardiac proteins or vesicle-derived nucleic acids captured in the second region. The platform supports release of intact vesicles, release of vesicle contents after lysis, or both, depending on the selected downstream assay.

Certain embodiments use the fractionated recovery arrangement. An early recovered fraction may be enriched in released vesicle contents, while later fractions contain lower concentrations or different classes of cardiac biomarkers. Common recovery may be used for multiplex analysis of surface-associated and internal vesicle biomarkers. Sequential recovery may be used so that intact sEVs, vesicle-associated proteins, and vesicle-derived internal biomarkers are recovered in different fractions.

In certain embodiments, the platform includes integrated signal-generation assemblies. One or more diagnostic signals are generated at the sampling assembly, within the matrix, at the enclosure, or in a coupled detection subsystem. The second set of cardiac-biomarker ligands may be used in a sandwich format with labeled detection antibodies or other labeled probes. Detection may be colorimetric, fluorescent, electrochemical, optical, impedance-based, or otherwise measurable. A first signal may correspond to intact sEV capture, and a second signal to released internal cardiac biomarkers, thereby allowing staged or multiplex interpretation of vesicle abundance and vesicle cargo.

Certain embodiments use computational logic to interpret one or more generated signals from intact sEVs, intravesicular cardiac proteins, vesicle-derived nucleic acids, or combinations. Normalized values, analyte ratios, weighted combinations, or composite cardiovascular indices may be calculated from one or more of CK-MB, troponin I, troponin T, myoglobin, CRP, Cystatin C, Cystatin S, BNP, NT-proBNP, hs-CRP, vesicle counts, or vesicle-derived nucleic acids. In embodiments, computational logic generates diagnostic interpretation.

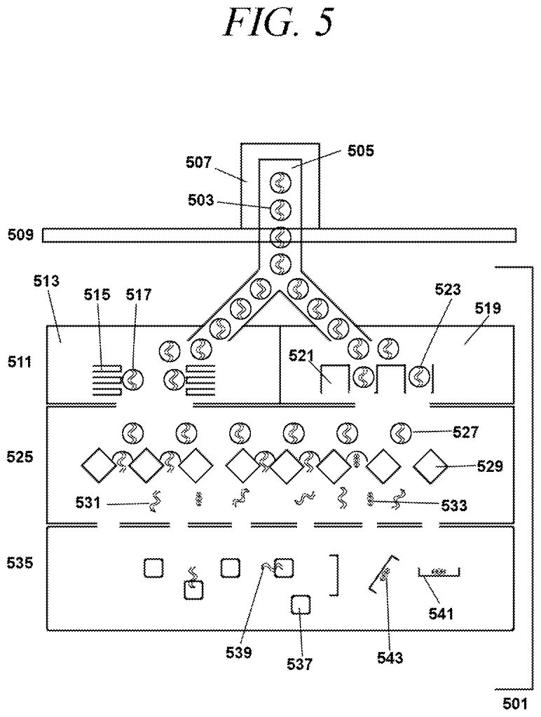

FIG. 5 illustrates a cardiovascular embodiment that captures and concentrates sEV-vesicles 503 and releases sEV-vesicle proteins 531 and optionally RNA or DNA 533 in a matrix 501 under an upstream conditioning region 509, the matrix including a first capture area 511, a vesicle-permeabilization region 525, and a downstream internal-cargo capture region 535.

The upstream conditioning region 509 is a thin upstream layer that reduces mucin and nonspecific background 507 before saliva 505 containing sEV-vesicles 503 reach the first capture area 511. The first capture area subregions include an sEV-binding chemistry capture subregion 513 that comprises different sEV-binding chemistries 515 that bind sEV-vesicles 517, and an sEV-surface-marker capture subregion 519 that comprises membrane-associated physicochemical or compositional features 521, such as antibodies or aptamers, peptides, lipid-interacting moieties, lectins, sulfated glycosaminoglycans, ion-exchange groups, charged polymer domains, or combinations, that bind sEV-vesicles 523. The vesicle-permeabilization region 525 subjects captured sEVs 527 to vesicle-opening processes 529 such as ionic or osmotic modulation, detergents or surfactants, thermal cycling, sonication, enzymatic membrane digestion, stimulus-activated lytic agents, or combinations, thereby exposing internal vesicle cardiac proteins 531 and optionally RNA or DNA 533. The downstream internal-cargo capture region 535 comprises a set of capture ligands 537 such as antibodies, aptamers, peptides, or other binders that are specific for intravesicular cardiac proteins 539 such as CK-MB, troponin I, troponin T, myoglobin, CRP, Cystatin C, Cystatin S, BNP, NT-proBNP, hs-CRP, or combinations, and optionally one or more nucleic-acid capture probes 541 to capture RNA or DNA 543. FIG. 5 shows that intact-vesicle capture, vesicle permeabilization, and internal-cargo capture occur as spatially differentiated functions within the same matrix.

Certain embodiments are useful for non-invasive cardiac screening, acute-event triage, coronary artery disease monitoring, heart-failure monitoring, risk stratification, prognosis determination, or longitudinal surveillance. Staged vesicle capture, staged internal-cargo release, and multiplex recovery of surface and internal biomarkers may provide a saliva-based alternative or complement to blood-based assessment of cardiovascular disease.

### 6. Diabetes

In certain embodiments, the diagnostic platform is configured to capture, concentrate, recover, and optionally detect salivary biomarkers associated with insulin resistance, pancreatic B-cell dysfunction, prediabetes, and diabetes mellitus. The biomarkers may include insulin, C-peptide, creatinine, glucose, or combinations. In embodiments, the platform is configured for multiplex detection of two, three, four, or more metabolic biomarkers within a single sampling event, thereby supporting metabolic classification and therapeutic monitoring from a single oral-fluid sample.

In certain embodiments, a volumetric sampling and concentration process enriches insulin and additional metabolic biomarkers from saliva or other biological fluids, and may improve detection of low-abundance insulin present at concentrations below 10 pg/mL, including concentrations in the range of about 1-5 pg/mL. The matrix may comprise one or more hydrogel, polyvinyl alcohol, cellulose-based, silica-based, hydrocolloid-based, or composite regions.

In certain embodiments, the platform includes a capture-configuration module with analyte-selective affinity domains arranged for multiplex metabolic assessment. Insulin and C-peptide may be captured using analyte-specific aptamers, analyte-selective molecularly imprinted polymers (MIPs), or hybrid architectures in which aptamers provide initial molecular recognition and MIP domains provide secondary high-capacity sequestration. Creatinine may be captured using ion-exchange groups, boronate chemistry, molecular imprinting, small-molecule affinity ligands, or combinations. Glucose may be captured using boronate-based affinity chemistry, glucose-binding proteins, glucose-specific aptamers, molecular imprinting, or combinations. In embodiments, capture domains for the different analytes are independently tunable with respect to ligand density, binding affinity, capacity, or combinations.

In certain embodiments, multiplex affinity domains are arranged within the matrix as co-immobilized domains in a common matrix, spatially segregated microdomains, layered structures, concentric architectures, stratified architectures, or combinations. Insulin has a molecular weight of approximately 5.8 kDa, substantially smaller than most circulating proteins. Due to its small size, insulin may exhibit limited diffusion into dense polymer networks. Small-analyte capture regions may be architected differently from creatinine and glucose regions so that insulin and C-peptide are captured in high-access regions while normalization-marker and metabolite regions are tuned for capacity, selectivity, or both.

Certain embodiments use the conditioned entry and biased routing arrangement, with an antifouling surface allowing diffusion of insulin, C-peptide, creatinine, glucose, or combinations toward the underlying capture regions. Insulin-selective domains may be positioned near the matrix surface beneath the antifouling layer to improve accessibility while limiting nonspecific fouling by salivary proteins and mucins. An active fluid-exchange and GCF-mobilization process may preload superabsorbent regions with aqueous fluid to reduce nonspecific adsorption, while selective capture chemistry compensates for dilution by concentrating target analytes within the matrix.

In certain embodiments, insulin-selective MIP domains are formed within or upon the matrix. Insulin possesses a highly distinctive and conserved molecular structure comprising two polypeptide chains linked by two interchain disulfide bonds and one intrachain disulfide bond, and further includes defined a-helical, B-sheet, and B-turn regions. This structural specificity enables effective molecular imprinting. Functional monomers may first be associated with insulin through hydrogen bonding, electrostatic interactions, hydrophobic interactions, or combinations before polymerization fixes the spatial arrangement of functional groups around the insulin template. Subsequent template removal leaves cavities complementary in size, shape, and chemical functionality to insulin. Poly(L-lysine)-based peptide crosslinkers undergo pH-induced helix-to-coil transitions that produce reversible swelling, and may thereby facilitate efficient insulin template removal as well as elution of captured insulin under mild aqueous conditions while preserving analyte integrity.

In certain embodiments, the diabetes embodiment uses reversible retention and release processes. All captured analytes may be eluted simultaneously into a common recovery volume, or sequential elution may be performed using differential release conditions so that insulin, C-peptide, creatinine, glucose, or combinations are quantified independently. In embodiments employing poly(L-lysine)-based crosslinkers, swelling-mediated release facilitates recovery of captured analytes under mild conditions.

Certain embodiments use the fractionated recovery arrangement to direct a selected recovered fraction toward detection while later-recovered fluid is diverted, withheld, or directed to waste. Metered recovery volumes may improve reproducibility of creatinine normalization, analyte-ratio comparison, and longitudinal interpretation across users and sampling events. Fractionated recovery may cooperate with multiplex capture so that the recovered fraction preserves informative relative levels of insulin, C-peptide, creatinine, glucose, or combinations. This may be accomplished automatically, with cartridge-coupled recovery processes.

In certain embodiments, the platform includes integrated signal-generation assemblies. Signals corresponding to insulin, C-peptide, creatinine, and glucose are generated as distinct signals or as portions of a composite signal.

Certain embodiments use computational logic to convert multiplex metabolic signals into one or more diagnostic outputs. Creatinine may serve as a normalization marker reflecting sample dilution, salivary flow rate, collection efficiency, or combinations. Insulin and C-peptide values may be normalized to creatinine concentration to improve quantitative accuracy and inter-sample comparability. Normalized insulin: C-peptide ratios and insulin: glucose ratios may be used to assess endogenous insulin secretion, exogenous insulin administration, insulin sensitivity, B-cell function, glycemic status, or combinations. Normalized insulin-to-creatinine, C-peptide-to-creatinine, and glucose-to-creatinine ratios may be calculated.

In certain embodiments, the computational logic accounts for volumetric concentration parameters of the platform, including biological fluid volume contacted during sampling, recovered fraction volume delivered for detection, estimated or measured concentration factor, multiplex analyte ratios, sampling duration, deformation cycles, elution volume, hydration state, or combinations. The computational logic may compensate for dilution of salivary biomarkers relative to corresponding serum levels and interpret concentrated salivary fractions in view of individualized baseline profiles, longitudinal trends, population-derived thresholds, or combinations. Outputs may include normoglycemia, insulin resistance, prediabetes, diabetes mellitus, trend indicators, severity indicators, therapeutic-response indicators, alerts, or recommendations for additional testing.

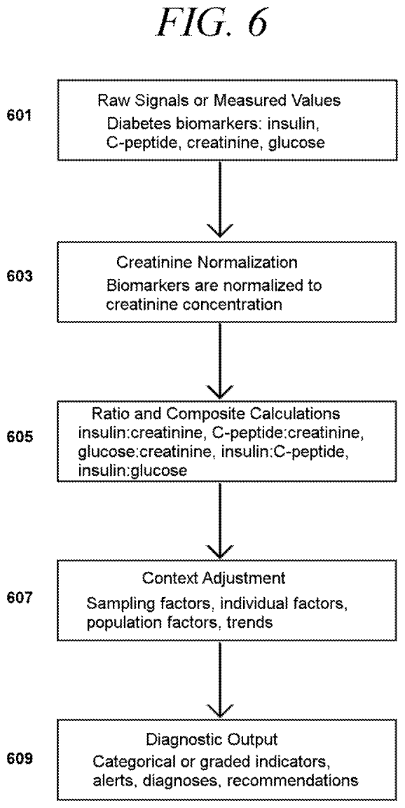

FIG. 6 is a computational and normalization flow diagram that illustrates an embodiment's computational logic phase. The phase begins with a step 601 where raw signals or measured values from insulin, C-peptide, creatinine, glucose enter processing. In a step 603, creatinine concentration serves as a normalization marker reflecting sample dilution, salivary flow rate, and collection efficiency, and insulin, C-peptide, and glucose values are normalized to creatinine concentration. In a step 605, ratio and composite calculations are made of insulin-to-creatinine, C-peptide-to-creatinine, glucose-to-creatinine, insulin: C-peptide, and insulin: glucose. In a step 607 context adjustment accounts for sampling duration, recovered volume, concentration factor, hydration state, baseline profile, longitudinal trends, population thresholds. In a step 609 diagnostic output reports on normoglycemia, insulin resistance, prediabetes, diabetes mellitus, trend, severity, therapeutic response, alert, recommendation for more testing.

Certain embodiments are useful for screening, early detection of insulin resistance, assessment of pancreatic B-cell reserve, classification of metabolic state, differentiation of endogenous insulin secretion from exogenous insulin administration, therapeutic monitoring, or longitudinal surveillance. The multiplex combination of insulin, C-peptide, creatinine, and glucose may distinguish the embodiment not merely by small-analyte capture, but by integrated normalization, ratio-based interpretation, and clinically useful downstream output generated from a single sampling event.

### 7. DNA Methylation Detection for Physiological Status and Disease Conditions

In certain embodiments, the diagnostic platform is configured with the volumetric sampling and concentration process to capture and concentrate DNA fragments and/or specific DNA sequences of interest for subsequent DNA methylation analysis. Methylation status at one or more CpG sites may be used as an epigenetic biomarker correlated with a physiological state, disease state, exposure history, treatment response, or disease progression. In embodiments, the target locus comprises cg05575921 at the AHRR locus, and relative hypomethylation at that locus is used as a marker of active smoking. In embodiments, salivary DNA methylation patterns at selected loci are evaluated for respiratory allergy monitoring, coeliac disease or other immune-related disorders, neuropsychiatric conditions including schizophrenia, depression, or anxiety disorders, or combinations. Longitudinal monitoring of salivary DNA methylation levels may be performed to assess treatment response, exposure change, or disease progression.

In certain embodiments, the DNA methylation embodiment uses the active fluid-exchange and GCF-mobilization process to enrich salivary DNA fragments, including cell-free DNA and other DNA-containing material accessible in saliva or saliva mixed with GCF. Matrix porosity and mesh size may be selected to permit diffusion and retention of DNA fragments while preserving access to tethered nucleic-acid capture ligands. The matrix may include a heterogeneous architecture with different regions tuned for DNA entry, methylation-state-selective hybridization, release, or combinations.

In certain embodiments, the platform includes a capture-configuration module with one or more nucleic-acid capture territories configured for methylation analysis. The matrix may comprise single-stranded oligonucleotide capture ligands, modified nucleic-acid probes, peptide nucleic acids, locked nucleic acids, affinity ligands, or combinations tethered to the matrix by flexible spacers including PEG spacers to improve target accessibility and hybridization kinetics. A first region may capture total or locus-specific DNA fragments prior to methylation-state discrimination. A second region may selectively capture DNA molecules corresponding to a converted methylated sequence, a converted unmethylated sequence, or both. In embodiments, the regions are arranged as adjacent regions, layered regions, cascaded regions, parallel lanes, or other spatially differentiated territories within the same matrix.

In certain embodiments, methylation-state-selective capture is achieved by converting cytosine methylation status into sequence differences prior to selective hybridization. Bisulfite conversion, enzymatic conversion, or another methylation-state-converting treatment may produce sequence divergence between methylated and unmethylated DNA at a target locus. Oligonucleotide capture ligands (probes) complementary to a converted methylated sequence may be positioned in one capture region, and probes complementary to a converted unmethylated sequence may be positioned in another capture region. The matrix may distinguish methylation state by sequence-selective capture after conversion rather than by direct recognition of methylcytosine alone. In embodiments, locus-specific capture is performed for one or more CpG-containing targets associated with smoking status, immune status, neuropsychiatric state, or other physiological conditions.

In embodiments, the capture region comprises probes which are generated as an overlapping set spanning one or more target loci, optionally on both strands, such that variable DNA fragments containing any of several targetable subsequences can hybridize to at least one probe. In embodiments involving methylation analysis, separate probe populations are generated for converted methylated and converted unmethylated sequence variants, with each probe including flanking sequence sufficient to support selective hybridization despite fragment-length variability. Converted methylated-sequence products and converted unmethylated-sequence products are not required to be independently pre-sorted before matrix entry, but instead diffuse through the matrix and are spatially separated by preferential hybridization and retention within different probe-bearing regions complementary to the respective converted sequence states.

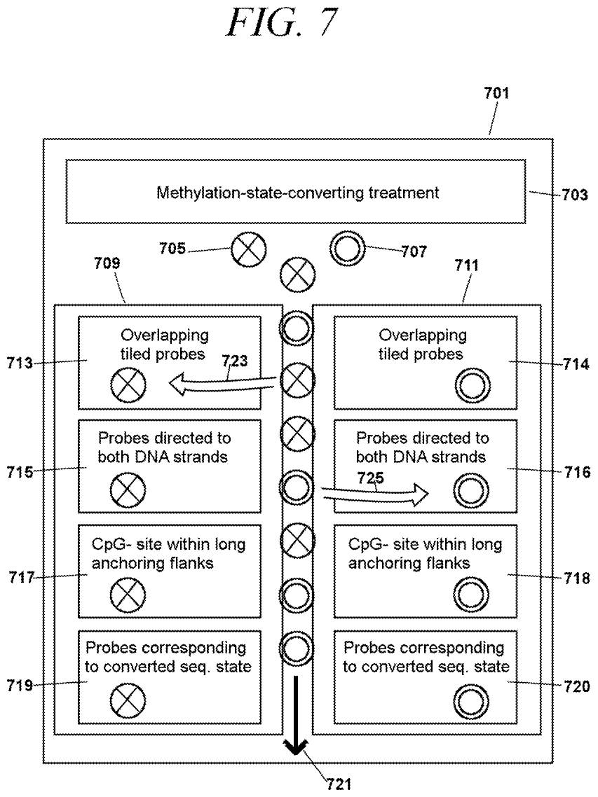

FIG. 7 illustrates a methylation-selective capture schematic in which a methylation-state-converting treatment 703 produces converted methylated-sequence products 705 and converted unmethylated-sequence products 707, which are captured in a methylated region 709 and an unmethylated region 711 of a matrix 701. Each region contains the same type of probe populations, in the methylated region arranged as overlapping tiled probes 713, probes directed to both DNA strands 715, probes in which the CpG-containing site is positioned within longer anchoring flanks 717, and probes complementary to the corresponding converted sequence state 719, and in the unmethylated region arranged as overlapping tiled probes 714, probes directed to both DNA strands 716, probes in which the CpG-containing site is positioned within longer anchoring flanks 718, and probes complementary to the corresponding converted sequence state 720. As the methylated-sequence products 705 and the unmethylated-sequence products 707 migrate 721 through the matrix 701, methylated-sequence products 705 hybridize selectively 723 within the methylated region 709, and unmethylated-sequence products 707 hybridize selectively 725 within the unmethylated region 711.

Certain embodiments use the conditioned entry and biased routing arrangement. An upstream conditioning region may enable salivary DNA to reach one or more downstream nucleic-acid capture territories where negatively charged DNA fragments concentrate. One or more DNA-focused capture regions may be positioned deeper within the matrix than surface conditioning structures so that DNA localization reflects fragment size, diffusivity, charge, hybridization kinetics, or combinations.

Certain embodiments use the reversible retention and release processes to release captured DNA. Release conditions may be selected to preserve suitability of the recovered eluate for methylation quantification by sequencing, PCR-based analysis, digital amplification workflows, probe-based readout, or combinations. A shallow or pathway-aligned capture arrangement may reduce rebinding so that released DNA is removed efficiently from the matrix before substantial rehybridization occurs.

Certain embodiments use the fractionated recovery arrangement to direct an initial recovered fraction toward downstream methylation analysis while later-recovered fluid is diverted, withheld, or directed to waste. An early recovered fraction may be enriched in locus-specific DNA relative to later fractions. Common recovery may be used when a single eluate is sent to sequencing or PCR. Sequential recovery may be used when one fraction is enriched for total captured DNA and another fraction is enriched for methylation-state-selective targets.

In certain embodiments, methylation-selective diagnostic signals may be generated after release of captured DNA to integrated signal-generation assemblies. In embodiments, the recovered DNA may be directed to a cartridge-coupled amplification or probe-based detection subsystem using cartridge-coupled recovery processes.

Certain embodiments use computational logic to interpret one or more locus-specific methylation signals. The computational logic may process methylated-sequence and unmethylated-sequence capture signals, normalized fractions, locus-to-locus comparisons, baseline comparisons, longitudinal trends, population-based thresholds, or combinations. The logic may generate one or more outputs including smoking-status classification, exposure-change indicator, disease-state classification, treatment-response indicator, progression assessment, alert, or recommendation for additional testing. In embodiments, repeated measurements from the same subject are compared over time to detect longitudinal methylation change at one or more target loci.

Certain embodiments are useful for non-invasive assessment of physiological status, disease conditions, environmental exposure, treatment response, or longitudinal biomarker change. The platform distinguishes itself not merely by salivary DNA capture, but by integrating DNA concentration, methylation-state conversion, sequence-selective hybrid capture, controlled release, and downstream analytical interpretation within a single oral-sampling architecture

### 8. HIV

In certain embodiments, the diagnostic platform is configured to capture, concentrate, recover, and optionally detect salivary biomarkers associated with HIV exposure or infection. The biomarkers may include HIV-specific IgG, IgA, IgM, or combinations. The platform may be configured to preferentially enrich salivary immunoglobulins before selective binding of HIV-specific antibodies, thereby improving sensitivity in a biological fluid in which antibody concentrations are typically lower than in blood and may vary depending on whether the sampled oral fluid is whole saliva, saliva enriched in GCF, or combinations.

Certain embodiments use the active fluid-exchange and GCF-mobilization process. The matrix donates fluid into the oral cavity during sampling, mixes with saliva and liberates GCF, then reabsorbs the mixed fluid, thereby increasing exposure of the matrix to HIV-associated antibodies present in saliva, GCF, or both. This process is especially useful for IgG-rich oral fluid derived in substantial part from serum transudation through gingival tissues. The same process may increase whole-mouth exposure of the matrix to locally produced IgA and other mucosal immunoglobulins.

HIV-associated antibody salivary concentrations are typically much lower than in blood, the dilution depending on the immunoglobulin isotype and the collection method (whole saliva vs oral fluid enriched in GCF). HIV IgG, which enters saliva mainly via GCF, is ~10²-10³ times lower than in blood. HIV IgA, secreted locally, is ~10¹-10² times lower than in blood. HIV IgM, whose large size minimizes transduction into saliva, is ~10³-10⁴ times lower than in blood. An affinity matrix may reduce or offset the effect of salivary dilution by concentrating HIV-relevant antibodies into a smaller diagnostically useful fraction with the volumetric sampling and concentration process. HIV-specific concentration architectures may achieve effective enrichment on the order of about 10³-10⁴ when a few milliliters of oral fluid are contacted and a substantially smaller recovered fraction is delivered for downstream analysis. Embodiments may preferentially target high-affinity IgG and concentrate the antibody signal.

In certain embodiments, the platform includes a capture-configuration module with spatially distinct antibody-handling regions arranged within the matrix. A first region may comprise immunoglobulin-binding agents that broadly capture and concentrate salivary IgG, IgA, and/or IgM. Immunoglobulin-binding agents may include anti-human IgG, IgA, and IgM antibodies, Fc-binding proteins or fragments, or combinations. A second region may comprise HIV antigens or antigenic fragments configured to bind HIV-specific antibodies enriched by the first region. Suitable HIV antigens may include gp41, gp120, gp160, gp24, their epitope peptides, conformational peptides, mimetic structures, or combinations. Regions may arranged as spatially distinct, overlapping, layered, or cascaded regions, or co-distributed regions within the same matrix.

In certain embodiments, salivary antibodies captured by immunoglobulin-binding agents subsequently move within and through the matrix to HIV-antigen binding sites by convection, deformation-induced transport, diffusion, or combinations. An active fluid-exchange and GCF-mobilization process liberates salivary antibodies, and cyclical compression, relaxation, chewing, or other oral mechanical loading may change pore geometry, interstitial fluid flux, hydraulic permeability, or combinations, increasing transport of captured or partially captured antibodies toward downstream HIV-antigen territories. Selective HIV capture may occur not merely by static diffusion, but by movement of antibody-enriched fluid within the matrix.

In certain embodiments, a conditioned entry and biased routing arrangement attracts biomarker-enriched oral fluid toward the sampling apparatus and into selected internal capture territories. Salivary conditioning and biomarker attraction may cooperate so that non-target macromolecules are reduced while antibody-rich oral fluid remains accessible to the capture regions. Electrostatic partitioning may increase movement of one or more HIV-associated antibodies, complexes, or GCF-borne immune components towards the sampling apparatus, relative to competing salivary species. Capillary-driven imbibition, deformation-responsive influx, and enclosure regions of differing porosity, wettability, thickness, charge, or permeability may recruit biomarker-enriched oral fluid from gingival margins, saliva pools, or both toward the sampling subsystem. Hydraulic transport, electrostatic partitioning, steric selectivity, and active fluid exchange increase delivery of HIV-relevant antibodies to the immunoglobulin-enrichment and antigen-capture regions.

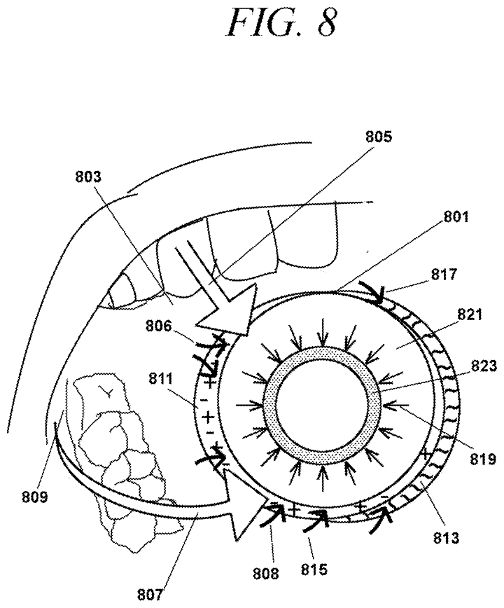

FIG. 8 schematically represents combined active fluid exchange, GCF mobilization, electrostatic recruitment, capillary recruitment, and inward transport toward immunoglobulin-enrichment regions within the same sampling apparatus, in an HIV embodiment. Saliva 805 moves from the oral cavity 803 toward a sampling apparatus 801, and GCF or GCF-enriched oral fluid 807 moves from gingival regions 809 toward the sampling apparatus 801. The sampling apparatus 801 may have a first peripheral region with electrostatic or ionic recruitment 811 and a second peripheral region with capillary recruitment 813. In embodiments, saliva-borne fluid fractions 806 and GCF-borne fluid fractions 808, one or both antibody-containing, are attracted toward one or both of the electrostatic region 811 and capillary region 813 before entering the matrix 821 at multiple locations. The electrostatic region 811 may recruit selected charged or charge-bearing biomolecular fractions 815, and the capillary region 813 may draw oral fluid with biomolecular fractions inward by capillary-active entry 817. Inward transport of recruited antibody-containing fluid 819 within the matrix 821 toward one or more immunoglobulin-binding regions 823 may occur prior to or during subsequent HIV-antigen-specific capture.

Certain embodiments use release systems described in the reversible retention and release processes. HIV-specific antibodies captured by antigen-bearing regions may be released by competitive displacement using soluble HIV antigens, soluble epitope peptides, or combinations.

Certain embodiments use the fractionated recovery arrangement to direct an initial recovered fraction enriched in HIV-associated antibodies toward detection while later-recovered fluid is diverted, withheld, or directed to waste. The recovered fraction may preserve information derived from both broad immunoglobulin enrichment and downstream HIV-antigen-specific capture. Selective recovery of an enriched fraction may improve sensitivity for oral HIV testing where target antibodies are initially dilute. The concentrated antibodies may remain structurally intact and suitable for downstream immunoassay, lateral-flow assay, ELISA, chemiluminescent assay, mass-spectrometric analysis, or combinations.

In certain embodiments, the platform includes integrated signal-generation assemblies to generate and locate signals. Generated signals are measurable, and may correspond to one or more of enriched IgG, enriched IgA, HIV-antigen-specific antibody binding, or combinations. Outputs support non-invasive detection of HIV exposure or infection and may distinguish mucosal and systemic immune-response components by relative IgA and IgG capture behavior.

Certain embodiments use computational logic to interpret one or more generated signals. The computational logic may accounts for known dilution of salivary antibodies relative to blood-derived antibody levels and interprets concentrated salivary fractions accordingly. In embodiments, the computational logic generates various relevant outputs.

Certain embodiments are useful for non-invasive screening, early-stage detection, chronic-infection monitoring, treated-infection monitoring, mucosal immune-response assessment, or longitudinal surveillance. The combination of active fluid exchange, GCF mobilization, biomarker-attracting transport bias, staged immunoglobulin enrichment, HIV-antigen-specific capture, and volumetric concentration provides a saliva-based diagnostic architecture that is stronger than any one of those elements alone.

### 9. Ovarian Cancer

In certain embodiments, the diagnostic platform is configured to capture, concentrate, recover, and optionally detect salivary biomarkers associated with ovarian cancer. The biomarkers may include CA-125, Human Epididymis Protein 4, Apolipoprotein A-I, or combinations. Multiplex assessment of a mucin-associated biomarker together with one or more soluble protein biomarkers may improve diagnostic specificity, sensitivity, or both, relative to reliance on a single biomarker alone.

Certain embodiments use the volumetric sampling and concentration process to enrich ovarian-cancer-associated biomarkers from saliva, saliva mixed with GCF, or both. Milliliter-scale sampling followed by microliter-scale elution supports multiplex concentration independent of biomarker identity.

In certain embodiments, the platform includes a capture-configuration module with spatially distinct ovarian-biomarker capture territories beneath a common antifouling surface layer. A CA-125 region may use oriented anti-CA-125 IgG or other epitope-specific affinity ligands within a shallow macroporous capture region positioned near the matrix surface. An HE-4 region may capture HE-4 using antibodies, aptamers, or combinations within a moderately porous structure. An ApoA-I region may employ lower ligand density or reduced capture-zone size to accommodate higher target abundance and reduce saturation. In embodiments, the regions are arranged as segregated zones, layers, parallel pathways, or mixed ligand territories, with each region tuned for pore size, ligand density, and fouling control.

Certain embodiments use the conditioned entry and biased routing arrangement. A strong antifouling layer may overlie one or more shallow capture territories and reduce interference from salivary mucins including MUC5B and MUC7, before CA-125, HE-4, or ApoA-I reach the capture regions. Shallow capture placement may preserve access for CA-125, which is a large, heavily glycosylated mucin-associated biomarker, while a more moderately porous HE-4 region and a separately tuned ApoA-I region may provide distinct transport and retention behavior for the smaller soluble proteins.

Certain embodiments use the reversible retention and release processes in a sequential differential-release format. ApoA-I may be released first using moderate salt or gentle pH change; HE-4 may be eluted next using mild low-pH or ionic adjustment, including aptamer strand displacement. CA-125 may be released last using brief low-pH glycine followed by rapid neutralization. In embodiments, all three biomarkers may instead be eluted into a common recovery volume when simultaneous multiplex analysis is preferred.

In certain embodiments, ApoA-I is captured in a region configured for earlier elution, by combining lower ligand density or reduced capture-zone volume with bait chemistries responsive to milder ionic-strength or pH shifts, optionally in a matrix architecture that limits deep penetration and repeated rebinding of ApoA-I, thereby permitting ApoA-I to be released before HE-4 and CA-125. The ApoA-I zone may be also associated with faster-clearing recovery geometry so that an initial elution step disproportionately removes ApoA-I relative to later-eluting biomarkers.

In certain embodiments, HE-4 is captured in a region configured for intermediate release. HE-4 release may be governed by reversible binding equilibrium and passive diffusion, such that unbound HE-4 diffuses from the matrix which shifts the equilibrium toward dissociation of additional bound HE-4. In embodiments, HE-4 is released by mild low-pH adjustment, ionic-strength increase, aptamer strand displacement, competing ligands, detergent exposure, chaotropic exposure, or combinations, depending on the selected bait chemistry. The relatively small size of HE-4 may permit penetration into and re-exit from the pore network, so that once dissociated, HE-4 can diffuse out of the matrix more readily than larger biomarkers.

In certain embodiments, premature release of HE-4 during an initial ApoA-I elution step is reduced by configuring the ApoA-I region with lower ligand density, reduced capture-zone volume, faster-clearing recovery geometry, or bait chemistry more responsive to a first ionic-strength or pH condition, while the HE-4 region employs a different ligand system, higher retentive density, delayed fluid access, or a second release trigger including aptamer strand displacement. The first elution step is therefore selective for the ApoA-I territory rather than merely mild in the abstract.

In certain embodiments, CA-125 is captured in a shallow, surface-accessible region configured for a large sterically constrained mucin-associated biomarker. The CA-125 region may employ oriented anti-CA-125 IgG or other epitope-specific ligands at low but highly accessible ligand density within a shallow macroporous structure beneath the antifouling layer. CA-125 may be retained through earlier ApoA-I and HE-4 elution conditions and may be released in a later step using brief low-pH glycine followed by rapid neutralization. Premature CA-125 release during an initial ApoA-I elution step may be reduced by directing the first elution condition preferentially through the ApoA-I capture territory, limiting first-step exposure time and recovered volume, and configuring the CA-125 region with a different release sensitivity such that substantial CA-125 release occurs only during a later brief low-pH glycine step followed by rapid neutralization. The CA-125 region may remain shallow for access during sampling but is protected from early release by differential routing, distinct ligand chemistry, or both.

In certain embodiments, the HE-4 region employs greater effective ligand density than the ApoA-I region, while the CA-125 region employs lower but highly accessible ligand density within a more porous macroporous structure, such that the three regions differ in retentive behavior and sequential elution response.

Certain embodiments use the fractionated recovery arrangement to support independent quantification of ApoA-I, HE-4, and CA-125. An initial fraction enriched in earlier-eluting ApoA-I may be routed toward a first collection region, a subsequent fraction enriched in HE-4 may be routed toward a second collection region, and a later fraction containing CA-125 may be routed toward a third collection region or common downstream detector. Recovery geometry and buffer sequence may cooperate with zone-specific release sensitivities to produce separate fractions without requiring complete analyte isolation at the capture stage.

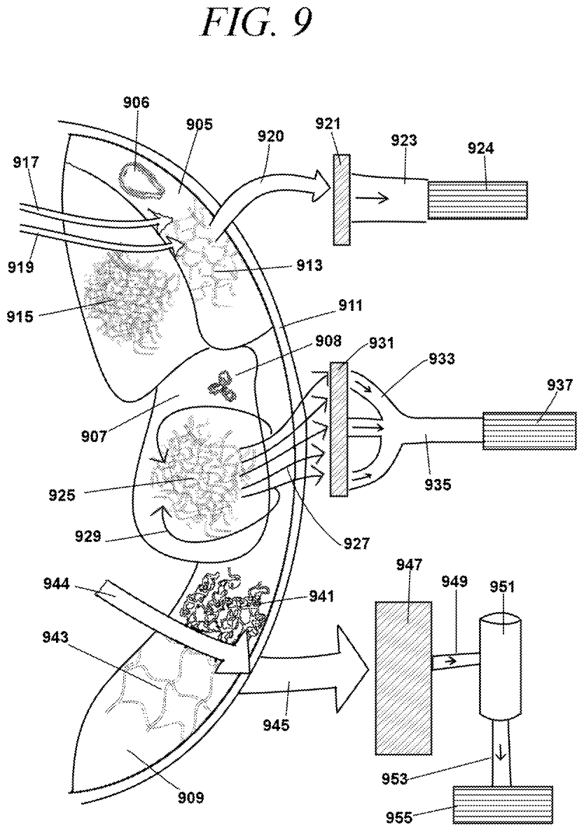

FIG. 9 illustrates certain embodiments of a staged elution and fractionated-recovery schematic for an ovarian-cancer embodiment. The matrix 901 includes spatially distinct ApoA-I territories 905, HE-4 territories 907, and CA-125 territories 909 are beneath a common antifouling layer 911, each territory having a different combination of pore architecture, ligand density, and release sensitivity. ApoA-I, a 28.1-28.3 kDa protein 906, in ApoA-1 territories 905, is configured for earlier elution by lower ligand density in lower mesh density 913 and reduced capture-zone volume, and a matrix architecture that positions higher mesh density in zone 915 below the ApoA-I territories 905, to limit deep penetration of ApoA-I 906. ApoA-I 906 release may be in response to mild ionic-strength 917 or pH 919 shifts, after which ApoA-1 906 is cleared 920 from the matrix 901, assisted by capillary pull from ApoA-1 wick 921. An early recovered, low-resistance fraction path 923 leads to detection zone 924. The HE-4 territories 907 have greater ligand density 925 relative to the other biomarker territories, and may be located at less shallow regions of the matrix, configured for intermediate release of HE-4 908, a 20-25 kDa glycoprotein. Passive diffusion 927 may lead to HE-4 dissociation by a reversible binding equilibrium, as diffusion feedback 929 shifts the equilibrium toward dissociation of additional bound HE-4 908. HE-4 wick 931 is more adjacent to HE-4 territories 907 than the ApoA-I wick 921 is adjacent to ApoA-1 territories 905. This facilitates outward diffusion 927 of HE-4 908. Release of HE-4 fluid from the wick 931 is rapid in multiple pathways 933, which clears fluid next to the HE-4 territories 907 to maintain the feedback 929 that allows more HE-4 908 to diffuse out. The HE-4 recovered fraction path 935 to an HE-4 detection zone 937 is a narrower, longer channel, relative to the ApoA-1 recovered fraction path 923 to the ApoA-1 detection zone 924, which permits ApoA-I 906 in recovered fraction path 923 to clear before HE-4 908 in recovered fraction path 935 clears.

The CA-125 territories 909 comprise a shallow macroporous region configured for steric access to CA-125 941, a very large glycoprotein, 200 kDa to over 5,000 kDa. The macroporous region has a large mesh size 943, resulting in lower but highly accessible ligand packing relative to the HE-4 mesh size 925 and the ApoA-I mesh size 913. CA-125 941 is held through the elution steps of ApoA-1 and HE-4, because the first elution conditions are made selective for the ApoA-I and HE-4 territories. CA-125 941 is then released using a brief flux of low-pH glycine 944, which drops local pH to to dissociate protein-protein interactions by weakening ionic interactions and hydrogen bonding. The CA-125 eluate moves on a capillary recovery path 945, driven by a large, strong capillary wick 947 (or another designed recovery-flow force), and the CA-125 recovered fraction path 949 leads to a neutralization column chamber 951 that contains an alkaline neutralization buffer, as rapid neutralization preserves the CA-125 fraction. The neutralization column chamber 951 has greater cross-sectional area than the recovered fraction path 949, and the chamber geometry provides sufficient mixing volume and residence time for rapid neutralization without excessive flow resistance. The chamber 951 output is a neutralized fraction path 953 to a CA-125 detection zone 955.

In certain embodiments, the platform includes integrated signal-generation assemblies. Separate signals may correspond to ApoA-I, HE-4, and CA-125, whether detected in sequentially recovered fractions or in a common multiplex readout. Detection may be colorimetric, fluorescent, electrochemical, optical, impedance-based, or otherwise measurable.

In certain embodiments, computational logic is used to interpret one or more generated signals from ApoA-I, HE-4, CA-125, or combinations. The multiplex value of the embodiment may arise not only from co-capture of biomarkers, but from controlled differential release and fraction-specific interpretation of biomarkers that differ greatly in abundance, steric accessibility, and release behavior.

The ovarian cancer embodiment may be useful for non-invasive screening, monitoring, follow-up after treatment, longitudinal surveillance, or triage for additional ovarian-cancer evaluation.

### 10. Tumors

In certain embodiments, the diagnostic platform is configured to capture, concentrate, recover, and optionally detect salivary glycoproteins associated with tumors, including breast cancer and other disease states in which aberrant glycosylation is present. The targeted biomarkers comprise glycoproteins bearing covalently attached carbohydrate moieties and are distinguished by glycan composition, cis-diol chemistry, charge characteristics, glyco-epitope structure, or combinations. Salivary tumor-associated glycoproteins may be enriched from saliva or other biological fluids for downstream biochemical or diagnostic analysis.

In certain embodiments, the volumetric sampling and concentration process enriches glycoprotein biomarkers from saliva, saliva mixed with GCF, or both. Matrix architecture may favor entry and retention of glycosylated proteins while reducing enrichment of non-glycosylated background species.

In certain embodiments, the platform includes a capture-configuration module with one or more glycoprotein-focused capture territories. A first territory may comprise lectin-functionalized capture ligands, a second territory may comprise boronic acid or boronate groups, and a third territory may comprise ion-exchange functional groups. One or more additional territories may comprise glyco-epitope antibodies, aptamers, or combinations directed to selected tumor-associated glycoproteins. The territories may be arranged as layered regions, parallel lanes, segregated domains, co-distributed microdomains, or combinations so that different glycoprotein classes encounter different capture chemistries within the same matrix.

In certain embodiments, lectin-functionalized regions comprise one or more lectins covalently tethered to the matrix, optionally through spacer groups that increase accessibility and binding efficiency. The lectins bind glycan motifs comprising mannose, glucose, fucose, sialic acid, N-acetylglucosamine, or combinations. Glycoproteins bearing complementary glycan structures may be retained in the lectin region while non-glycosylated proteins are relatively excluded.

In certain embodiments, boronate-functionalized regions comprise boronic acid or boronate groups incorporated into the matrix backbone or tethered through spacer molecules. These regions reversibly bind cis-diol-containing carbohydrate structures on glycoproteins through diol-boronate interactions. Boronate-based capture may be used alone or with lectin-based capture to enrich glycoproteins differing in glycan structure, abundance, or reversibility of binding.

In certain embodiments, ion-exchange regions comprise cation-exchange groups, anion-exchange groups, or both. Glycosylation alters net charge, hydration, isoelectric behavior, or combinations, thereby permitting preferential enrichment of tumor-associated glycoproteins under selected pH and salt conditions. Ion-exchange capture may be used alone or in combination with lectin or boronate capture so that glycoproteins are enriched by both charge-dependent and glycan-dependent interactions.

Certain embodiments use the conditioned entry and biased routing arrangement. An upstream conditioning region may enable salivary glycoproteins to reach one or more downstream capture territories. One or more regions may be configured so that glycoproteins are preferentially directed toward lectin, boronate, ion-exchange, glyco-epitope antibody, or aptamer territories according to glycan accessibility, charge, hydration, steric behavior, or combinations. Selective routing and selective capture may cooperate to enrich tumor-associated glycoproteins relative to non-glycosylated or less diagnostically useful salivary proteins.

Certain embodiments employ combined and multiplex capture. Lectin-based binding agents, boronate groups, ion-exchange functional groups, glyco-epitope antibodies, aptamers, or combinations may be distributed uniformly, spatially, or functionally throughout the matrix to increase selectivity, capacity, robustness, or combinations for enrichment of glycoproteins associated with disease states. Different glycoprotein classes may be captured by different chemistries within the same matrix so that the resulting eluate carries information about both abundance and glycan class.

In certain embodiments, the reversible retention and release processes is used. Glycoproteins captured in lectin regions may be released by competitive displacement using free carbohydrates, by pH modulation, by ionic-strength adjustment, or by cleavage of a reversible linker coupling the lectin to the matrix. Glycoproteins captured in boronate regions may be released by pH modulation, ionic-strength adjustment, competitive diol-containing compounds, or combinations. Glycoproteins captured in ion-exchange regions may be released by adjusting pH or salt concentration so as to weaken electrostatic interactions. Release conditions may be selected to generate an eluate suitable for downstream tumor-marker analysis.

In certain embodiments, the fractionated recovery arrangement is used. A recovered fraction may be enriched in glycoproteins captured through a first chemistry and a later fraction may be enriched in glycoproteins captured through a second chemistry. A first fraction may be enriched in lectin-captured glycoproteins, a second fraction in boronate-captured glycoproteins, and a third fraction in ion-exchange-enriched glycoproteins, or all captured glycoproteins may be released into a common multiplex recovery volume. Staged recovery may preserve diagnostically useful differences between glycoprotein classes and capture mechanisms.

In certain embodiments, the platform includes integrated signal-generation assemblies. Different signals may correspond to different glycoprotein capture chemistries, different glycan classes, different tumor-associated glycoproteins, or combinations. Detection may be colorimetric, fluorescent, electrochemical, optical, impedance-based, or otherwise measurable.

Certain embodiments use computational logic to interpret one or more generated signals from lectin-based, boronate-based, ion-exchange-based, glyco-epitope-based, or aptamer-based capture regions. The platform may derive diagnostic value not only from capture of a single tumor marker, but from multiplex characterization of aberrant salivary glycoprotein content across multiple reversible capture chemistries.

Certain embodiments may be useful for screening, monitoring, follow-up, longitudinal surveillance, or triage for additional evaluation of tumors associated with altered salivary glycoproteins. The combination of lectin capture, boronate capture, ion-exchange enrichment, and optional glyco-epitope-specific capture may provide a saliva-based tumor-glycoprotein workflow that differs from a single-ligand assay by enriching multiple glycoprotein classes through distinct but coordinated capture and release mechanisms.

### 11. Parkinson's Disease

In certain embodiments, the diagnostic platform is configured to capture, concentrate, recover, and optionally detect salivary a-synuclein associated with Parkinson's disease and related synucleinopathies. The targeted biomarker comprises total a-synuclein, oligomeric or aggregated a-synuclein, post-translationally modified a-synuclein, extracellular-vesicle-associated a-synuclein, or combinations.

In certain embodiments, the volumetric sampling and concentration process is used to enrich salivary a-synuclein from saliva or saliva mixed with GCF. The matrix may be modified with antibodies, affinity ligands, peptides, aptamers, MIPs, or combinations configured to bind one or more a-synuclein forms. Saliva may enter the matrix through the enclosure, a-synuclein is absorbed and retained, and the retained biomarker is subsequently released by one or more release mechanisms described herein, including, where appropriate, vesicle permeabilization for extracellular-vesicle-associated a-synuclein.

In certain embodiments, the platform includes integrated signal-generation assemblies or a coupled detection subsystem configured to analyze released a-synuclein for screening, monitoring, or research related to Parkinson's disease. Computational logic may evaluate one or more a-synuclein signals, forms, or relative abundances to generate a classification, trend indication, progression assessment, or other diagnostic interpretation.

### 12. Oral Squamous Cell Carcinoma, Lupus, Arthritis, Sjogren's Syndrome

In certain embodiments, the diagnostic platform is configured to capture, concentrate, recover, and optionally detect salivary autoantibodies associated with oral squamous cell carcinoma, systemic lupus erythematosus, rheumatoid arthritis, Sjogren's syndrome, or related autoimmune or inflammatory conditions. Targeted biomarkers comprise disease-associated autoantibodies of one or more isotypes including IgA, IgG, IgM, or combinations. The platform may be configured for diagnostic classification, inflammatory-status assessment, flare prediction, longitudinal monitoring, or combinations.

Certain embodiment uses the volumetric sampling and concentration process to enrich disease-associated autoantibodies from saliva, saliva mixed with GCF, or both.

In certain embodiments, the platform includes a capture-configuration module in which one or more autoantigens or other affinity ligands are immobilized within spatially defined capture territories of the matrix. Polymer chains or other matrix components may bear reactive functional groups capable of forming stable covalent bonds with complementary functional groups on antigens. The antigens may be presented as full-length proteins, protein domains, peptide epitopes, or combinations, and preferentially capture disease-associated autoantibodies entering the matrix from biological fluid. Different capture territories may present different autoantigens or epitope sets so that the resulting autoantibody profile reflects disease-specific or flare-associated binding patterns rather than a single undifferentiated signal.

Certain embodiments use the conditioned entry and biased routing arrangement. Such routing may improve enrichment of diagnostically useful autoantibody classes relative to non-target salivary components.

In certain embodiments, binding of autoantibodies to immobilized antigens is reversible and the embodiment uses the reversible retention and release processes. The recovered fraction may preserve the disease-associated autoantibody profile for downstream analysis in a coupled detection subsystem.

In certain embodiments, the embodiment uses the fractionated recovery arrangement to deliver an enriched autoantibody fraction to detection. A selected recovered fraction may be directed to a detection region while later fluid is diverted, withheld, or directed to waste.

In certain embodiments, the platform includes integrated signal-generation assemblies configured to generate one or more signals corresponding to captured autoantibodies or recovered autoantibody fractions in the sampling apparatus. Different autoantigen-presenting territories may produce distinguishable signals, thereby generating an autoantibody profile rather than a single binary result.

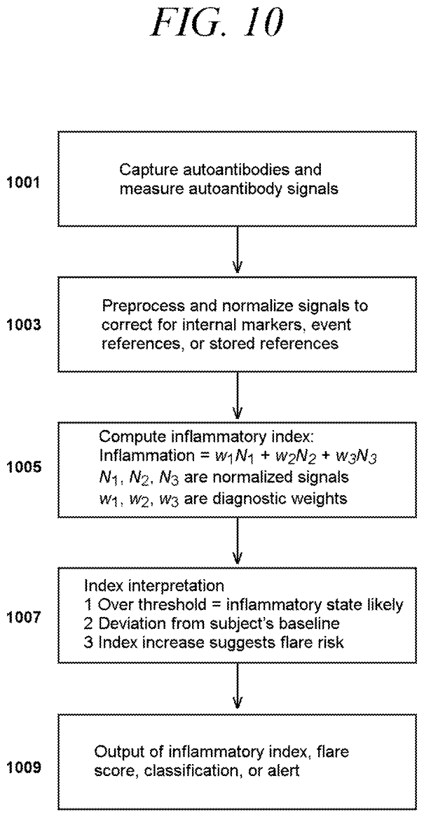

Certain embodiments use computational logic to convert disease-associated autoantibody signals into one or more inflammatory indices, flare prediction scores, classifications, alerts, severity indications, trend indications, or other diagnostic interpretations, following a staged workflow illustrated in FIG. 10. In stage one 1001, disease-associated autoantibodies are captured and measured to generate one or more autoantibody signals. In stage two 1003, the signals are preprocessed and normalized to generate normalized signals corresponding to a different autoantigen territory, autoantibody class, or disease-associated binding pattern. Preprocessing and normalization correct for one or more of internal reference markers, same-event reference values, recovered volume, concentration factor, sample volume, elapsed sampling time, hydration state, prior measurements from the same subject, longitudinal trends, stored threshold values, population-based profiles, external reference standards, or combinations. In stage three 1005, normalized signals such as N₁, N₂, N₃, each from different autoantigen territories, are determined. Diagnostic weights such as w₁, w₂, w₃, using regression-derived values, multivariate scores, or composite indices, are determined. The weights may be fixed, empirically derived, subject-specific, disease-specific, or combinations. An inflammatory index is produced as a weighted combination of the normalized signals, such as a sum of the form Inflammation=w₁N₁+w₂N₂+w₃N₃. In stage four 1007, the inflammatory index interpretation includes determining whether the computed inflammatory index exceeds one or more stored thresholds, whether the computed index deviates from an individualized baseline profile for the same subject, and whether the computed index has increased relative to one or more prior measurements so as to indicate rising flare risk. These three forms of interpretation, threshold-based interpretation, baseline-deviation interpretation, and longitudinal-increase interpretation, may be used individually or in combination to classify inflammatory status, predict flare, or generate an alert. In stage five 1009, the resulting inflammatory index or flare prediction score determines whether the observed autoantibody pattern indicates stable status, increased inflammatory activity, likely flare, progression, treatment response, or need for additional testing. Outputs may include a raw or normalized index, categorical classification, flare risk category, alert, severity scale, longitudinal trend indicator, therapeutic-monitoring output, or combinations.

In certain embodiments, the embodiment is useful for screening, diagnosis, disease-activity assessment, flare monitoring, therapeutic monitoring, or longitudinal surveillance in oral squamous cell carcinoma, lupus, arthritis, Sjogren's syndrome, or related conditions in which salivary autoantibody patterns are informative. In embodiments, the value of the embodiment arises not only from autoantibody capture, but from generation and interpretation of an autoantibody profile through computational logic configured to compute inflammatory indices and flare prediction scores.

### Onboard Diagnostic Displays

The following two embodiments illustrate two different ways that the sampling apparatus may be used as the location for diagnostic analysis and diagnostic displays. The first embodiment is for Sjogren's syndrome, and shows how a multiplex detection arrangement and display can be configured entirely within the matrix. The second embodiment is for a measles diagnostic platform, in which the matrix contents are not eluted for detection, but rather the matrix itself serves as the backbone for in situ RNA diagnosis.

### 13. Sjogren's Syndrome

In certain embodiments, the diagnostic platform is configured to capture, concentrate, recover, and optionally detect salivary biomarkers associated with Sjogren's syndrome. The biomarkers comprise one or more metabolites including lactate, alanine, and taurine, one or more proteins including NGAL, B2-microglobulin, annexin A2, siglec-5, and CA-VI, one or more regulatory RNAs, or combinations. The platform is configured for multiplex detection of two, three, four, or more Sjogren's-associated biomarkers within a single oral-fluid sampling event. The biomarker selection is consistent with recent salivary-biomarker reviews reporting repeated elevation of those analytes in Sjogren's syndrome.

The Sjogren's embodiment uses the volumetric sampling and concentration process to enrich salivary biomarkers from saliva, saliva mixed with GCF, or both. The active fluid-exchange and GCF-mobilization process is especially useful in Sjogren's syndrome because salivary flow is often reduced and biomarker concentration may vary with dryness severity and gland dysfunction. Reduced salivary flow is a recognized feature of Sjogren's disease.

In certain embodiments, the platform includes a capture-configuration module with different biomarker-handling regions for different analyte classes. A first region may be configured for small metabolites including lactate, alanine, and taurine; a second region may be configured for proteins including NGAL, B2-microglobulin, annexin A2, siglec-5, and CA-VI; and a third region may be configured for regulatory RNAs. The metabolite region comprises one or more enzyme-coupled, aptamer-based, molecularly imprinted, or other small-analyte recognition chemistries; the protein region comprises antibodies, antibody fragments, aptamers, peptides, or combinations; and the regulatory RNA region comprises sequence-selective nucleic-acid capture ligands. These regions may be arranged as adjacent regions, layered regions, parallel lanes, or other spatially differentiated territories within the same matrix. In certain embodiments, the Sjogren's embodiment uses a conditioned entry and biased routing arrangement to reduce mucin fouling and improve delivery of salivary biomarkers to their corresponding capture territories.

The platform may be configured to include integrated signal-generation assemblies positioned at the sampling apparatus so that results are reported without transfer of the matrix to a separate post-sampling device. One or more metabolites are measured by colorimetric, fluorescent, electrochemical, or optical reporter chemistries generated within the matrix or at the enclosure. One or more proteins are measured by sandwich-type or direct-binding immunodetection using labeled antibodies, aptamers, enzymes, fluorophores, nanoparticles, electrodes, or combinations. One or more regulatory RNAs are measured by sequence-selective hybridization with labeled probes, probe-mediated amplification, fluorescence generation, electrochemical signal generation, or combinations. The signal-generating elements may be integrated into the sampling assembly so that metabolite, protein, and RNA results are produced and read at the sampling apparatus itself rather than after transfer to an external cartridge or analyzer.

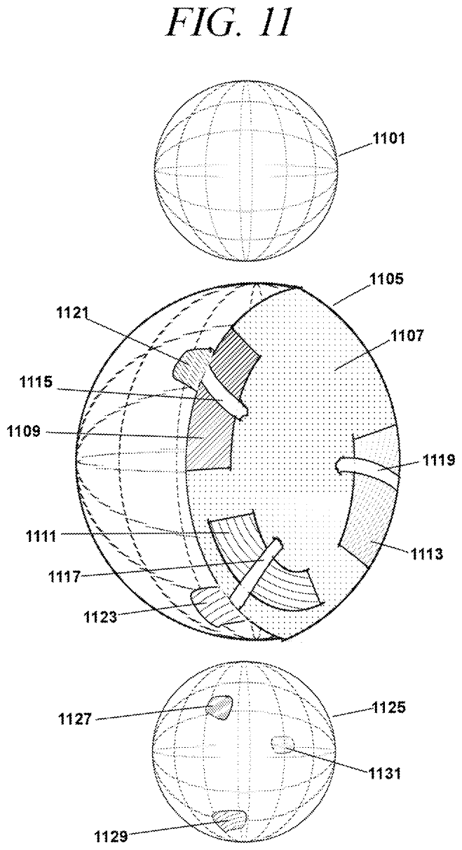

FIG. 11 illustrates a sampling apparatus for a Sjogren's syndrome embodiment in which salivary biomarkers are detected and reported in situ at the sampling apparatus. At top is a matrix 1101 configured to be placed in a fluid-permeable enclosure for use in an oral cavity. In the middle is a cutaway view 1105 of the matrix, showing the internal matrix 1107 configured to receive saliva. Positioned in the matrix 1107 is a Sjogren's-associated metabolites capture region 1109, a Sjogren's-associated protein capture region 1111, and a Sjogren's-associated regulatory RNA capture region 1113. A metabolites signal-generating region 1115 is within the metabolites capture region 1109, a protein signal-generating region 1117 is within the protein capture region 1111, and a regulatory RNA signal-generating region 1119 is within the protein capture region 1113. These signal-generating regions generate biomarker-dependent signals without transfer of the matrix to a separate post-sampling device. Visible on the matrix exterior surface are Sjogren's-associated metabolites signals 1121, Sjogren's-associated protein signals 1123, and Sjogren's-associated regulatory RNA signals. At bottom the matrix 1125 is shown after signal generation, with metabolites signals 1127, protein signals 1129, and, as seen through the matrix, regulatory RNA signals 1131.

The result-display regions in the matrix provide a visual, fluorescent, electrochemical, optical, or other readable indication corresponding to one or more elevated Sjogren's-associated biomarkers or to a multiplex diagnostic output derived from them.

In certain embodiments, the platform generates a multiplex Sjogren's profile in which elevated lactate, alanine, taurine, NGAL, B2-microglobulin, annexin A2, siglec-5, CA-VI, regulatory RNAs, or combinations are measured together. The measured signals may be interpreted by computational logic to produce a Sjogren's-associated classification, dryness-associated severity output, inflammatory-state output, longitudinal trend indicator, or recommendation for additional testing. The computational logic uses normalized signal values, analyte ratios, baseline comparison, prior measurements from the same subject, longitudinal trends, or combinations. The logic may weight protein, metabolite, and RNA markers differently according to their diagnostic relevance.

In certain embodiments, the Sjogren's embodiment is useful for non-invasive screening, aid in diagnosis, longitudinal monitoring, or treatment-response monitoring in subjects with suspected or established Sjogren's syndrome. Salivary metabolomics and proteomics studies have repeatedly shown elevated lactate and alanine, and broader reviews support the promise of salivary protein and RNA panels for Sjogren's diagnosis, although current literature still emphasizes the need for validation before any single salivary marker panel is treated as definitive clinical standard of care.

### 14. Measles

In certain embodiments, the diagnostic platform is configured to capture, concentrate, recover, and optionally detect salivary biomarkers ly nucleic-acid result is obtained from oral fluid without venipuncture, while a later or parallel serologic result is obtained from the same or a related oral-fluid sampling event. Detection of measles-specific IgM associated with measles infection. The targeted biomarkers comprise measles virus RNA, measles-specific IgM antibodies, or combinations thereof. The platform may be configured so that an ear in serum remains a standard laboratory confirmation approach, while measles RNA testing is widely performed on respiratory specimens collected as soon as measles is suspected; WHO materials further recognize oral fluid as a validated specimen type for measles IgM when an oral-fluid-validated assay is used, and as a good source of measles RNA for molecular detection. Oral fluid has also been shown experimentally to support both measles IgM and measles RNA testing, with one study reporting oral-fluid IgM sensitivity/specificity of 92%/100% and oral-fluid measles RNA sensitivity/specificity of 100%/100% against serum and nasopharyngeal comparators.

Certain embodiments use the volumetric sampling and concentration process to enrich measles-associated biomarkers from saliva, saliva mixed with GCF, or both. The matrix is retained by a fluid-permeable enclosure configured for oral use and contacts a larger oral-fluid volume during sampling while yielding a smaller diagnostically useful fraction or localized amplification-ready fraction. This non-invasive sampling format is especially useful for pediatric subjects, field screening, outbreak response, and early triage, where rapid oral-fluid collection may be easier than blood collection and better tolerated than throat or nasopharyngeal swabbing.

In certain embodiments, the platform includes a capture-configuration module with at least a first nucleic-acid-focused region and, optionally, a second antibody-focused region. The nucleic-acid-focused region comprises one or more charged polymer domains, ion-exchange groups, cationic microdomains, RNA-accessible hydrogel domains, or combinations that increase local availability of negatively charged measles RNA or measles-RNA-containing particles to an amplification-compatible matrix region. The matrix may further include a conditioned entry and biased routing arrangement that reduces mucin fouling and partitions oral fluid so that RNA-containing fluid fractions preferentially reach the nucleic-acid region. A parallel or downstream antibody-focused region may include immunoglobulin-binding agents and measles-antigen-presenting capture ligands configured to enrich or detect measles-specific IgM from oral fluid, thereby permitting a dual-mode oral-fluid measles assay in which RNA and antibody information are obtained from the same platform architecture. WHO guidance indicates that oral-fluid IgM testing should use an oral-fluid-validated assay, and CDC recommends collecting both serology and molecular specimens at first contact with a suspected case.

Certain embodiments employ a diagnostic method in which biological fluid is not eluted from the matrix. Rapid measles RNA detection occurs within the matrix itself by hydrogel-mediated reverse transcription loop-mediated isothermal amplification (hLAMP) without nucleic-acid extraction. After oral-fluid sampling the matrix is placed into a sealed cartridge for an isothermal amplification interval of about 30 minutes. Cartridge closure may bring the matrix into functional association with one or more reagent reservoirs containing reverse-transcription LAMP reagents and measles-specific primers that target one or more measles genomic targets, buffer, and optional detection reagents, or the reagents are prepositioned within the hydrogel before use. Cartridge closure may slice the matrix hydrogel onto a chip. The matrix hydrogel may be heated for digital isothermal amplification so that measles RNA is amplified in situ without a separate nucleic-acid extraction step.

The platform includes integrated signal-generation assemblies configured to report hLAMP amplification within the cartridge. Signal generation may be colorimetric, fluorescent, optical, electrochemical, or otherwise measurable through an optical window or reader interface in the cartridge. The same cartridge optionally contains a second detection region for measles-specific IgM, including a lateral-flow region, optical antibody-detection region, or other reporter subsystem, so that early nucleic-acid detection and later serologic confirmation are both supported by the same platform. Computational logic distinguishes at least three clinically useful states: (i) measles RNA detected with no or low measles-specific IgM, consistent with very early infection; (ii) measles RNA detected with measles-specific IgM, consistent with active infection around or after rash onset; and (iii) measles RNA not detected with measles-specific IgM detected, consistent with a later serologic window or resolving infection, subject to correlation with timing and clinical context. CDC notes that measles RNA testing has greatest sensitivity when respiratory specimens are collected at first contact, whereas serum IgM may be negative in some cases until about 3 days after symptom onset; the oral-fluid embodiment uses those timing differences as a design rationale for combined RNA-plus-IgM testing.

All numbers expressing quantities, percentages, or proportions are to be understood as being modified by the term “about” unless otherwise indicated. Ranges may be expressed as from one value to another, and such ranges are intended to include all subranges therebetween. The disclosure of individual values serves as a disclosure of the entire range and all subranges.

As used herein, the singular forms “a,” “an,” and “the” include plural referents unless the context clearly dictates otherwise. The term “comprising” is intended to be open-ended and includes the term “consisting essentially of” and “consisting of.” All technical and scientific terms used herein have the same meaning as commonly understood by one of ordinary skill in the art.

The scope of the invention should not be limited by the particular embodiments described herein, but should be defined only by the appended claims and their equivalents. Where a means or step is recited in the claims without specifying structure, it is intended that the recitation be interpreted broadly, consistent with 35 U.S.C. 112(f) (pre-AIA 112, sixth paragraph).

All publications, patents, and patent applications cited in this specification are hereby incorporated by reference in their entirety for all purposes as if each individual publication or patent were specifically and individually indicated to be incorporated by reference.

Accordingly, the invention is not to be limited except as by the appended claims.

1. A diagnostic system configured to generate a detectable signal corresponding to one or more biomarkers in a biological fluid, the system comprising:
a sampling subsystem comprising a fluid-accessible sampling matrix positioned within a fluid-permeable enclosure; and
a detection subsystem comprising at least one signal-generating element configured to generate the detectable signal corresponding to the one or more biomarkers;
wherein the sampling subsystem is configured to preferentially increase a quantity of the one or more biomarkers bound to one or more immobilized capture domains in the sampling matrix relative to at least one other biological compound present in the biological fluid by at least one of:
(a) a migration-biasing structure configured to generate a transport-driving gradient that produces differential migration of the one or more biomarkers relative to other biological compounds prior to binding by the one or more immobilized capture domains; or
(b) the fluid-accessible sampling matrix being heterogeneous and including a plurality of capture regions each presenting one or more immobilized capture domains, the heterogeneous fluid-accessible sampling matrix arranged such that biological fluid is differentially exposed to the plurality of capture regions, the plurality of capture regions selectively capturing at least one of the one or more biomarkers relative to other biological compounds;
and wherein a volume of biological fluid contacted by the sampling subsystem during operation exceeds a volume of fluid delivered to the detection subsystem by at least one order of magnitude.

2. The diagnostic system of claim 1, wherein the sampling matrix comprises at least two spatially distinct capture regions presenting immobilized capture domains, the capture regions arranged in one or more of:
a layered configuration,
radial zoning,
axial zoning,
parallel flow paths, or
cascaded capture zones,
wherein the at least two capture regions differentially influence at least one of biomarker attraction, transport, binding, retention, or elution.

3. The diagnostic system of claim 1, wherein a conditioning layer is positioned between the fluid-permeable enclosure and the sampling matrix, the conditioning layer configured to reduce mucin fouling, particulate interference, or nonspecific adsorption prior to the biological fluid reaching at least one of the plurality of capture regions and to bias movement of the biological fluid toward selected capture regions.

4. The diagnostic system of claim 1, wherein the heterogeneous sampling matrix comprises both affinity ligands and chemically interactive functional groups arranged in spatially distinct capture regions.

5. The diagnostic system of claim 1, wherein directing biological fluid enriched in the one or more biomarkers toward the sampling subsystem comprises:
a migration-biasing structure disposed adjacent to and in fluid communication with the sampling matrix, the migration-biasing structure comprising at least one of:
(i) a charged region comprising fixed ionic functional groups configured to establish an electrostatic potential gradient relative to the biological fluid; or
(ii) a capillary-accessible surface geometry configured to generate a capillary pressure differential;
wherein the migration-biasing structure alters the composition of biological fluid reaching a capture region of the sampling matrix body by increasing a local concentration of the one or more biomarkers relative to other biological compounds prior to binding by the immobilized capture domain.

6. The diagnostic system of claim 2, wherein at least two of the plurality of capture regions comprise different materials selected from inorganic, organic, polymeric, hybrid, or composite materials.

7. The diagnostic system of claim 1, wherein at least one of the plurality of capture regions comprises a nucleic-acid capture region having a first subregion comprising oligonucleotide probes complementary to a converted unmethylated target sequence and a second subregion comprising oligonucleotide probes complementary to a converted methylated target sequence.

8. The diagnostic system of claim 7, wherein the nucleic-acid capture region further comprises one or more charged polymer domains, ion-exchange groups, cationic microdomains, or electrostatically interactive regions configured to bias negatively charged nucleic acids or nucleic-acid-containing particles toward the first and second subregions.

9. A diagnostic platform configured to generate one or a plurality of diagnostic signals corresponding to one or more biomarkers in a biological fluid, the platform comprising:
a sampling system comprising:
a sampling matrix including at least one capture region presenting at least one immobilized capture domain capable of binding the one or more biomarkers; and
a fluid-permeable enclosure positioned relative to the sampling matrix so as to retain the sampling matrix during sampling, the enclosure comprising at least one fluid-permeable region configured to permit exchange of biological fluid between an external environment and the sampling matrix; and
one or more signal-generating elements integrated into or onto the sampling system and configured to generate the one or plurality of diagnostic signals based on the one or more biomarkers associated with the sampling matrix, wherein the one or more signal-generating elements comprise at least one of:
(i) sensing elements integrated into or onto the sampling matrix;
(ii) sensing elements integrated into or onto the fluid-permeable enclosure; or
(iii) signal-generating elements introduced into contact with the sampling matrix after biomarker binding.

10. The diagnostic platform of claim 9, wherein the sampling matrix is configured as an in-matrix assay region such that the one or more biomarkers are subjected to loop-mediated isothermal amplification, reverse-transcription loop-mediated isothermal amplification, immunoreaction-based signal development, enzyme-mediated color generation, fluorescence generation, electrochemical analysis, or a combination thereof, without prior extraction of the one or more biomarkers from the sampling matrix.

11. The diagnostic platform of claim 10, wherein the sampling system is configured to be inserted into a cartridge or housing after sampling, and wherein diagnostic analysis occurs in the sampling matrix without prior extraction of the one or more biomarkers from the sampling matrix.

12. The diagnostic platform of claim 9, wherein the detection arrangement comprises at least one of:
(i) at least one electrode positioned in electrical communication with the sampling matrix and configured to generate an electrical signal corresponding to the one or more biomarkers; or
(ii) an optical detection region configured to detect fluorescence, colorimetric change, luminescence, or reflectance generated within the sampling matrix.

13. The diagnostic platform of claim 9, wherein the signal-generating elements comprise at least one of:
(i) affinity reagents configured to bind to the one or more biomarkers bound within the sampling matrix;
(ii) one or more enzymes configured to react with the one or more biomarkers to produce an optical or electrochemical signal within the sampling matrix; or
(iii) nucleic acid amplification or CRISPR-based reagents configured to generate a detectable signal upon interaction with a nucleic acid biomarker within the sampling matrix.

14. A method of generating a detectable signal corresponding to at least one biomarker in a biological fluid, comprising:
a) contacting the biological fluid with a sampling subsystem comprising a fluid-accessible sampling matrix including one or more capture regions presenting at least one immobilized capture domain capable of binding the at least one biomarker;
b) binding at least a portion of the at least one biomarker within the sampling matrix;
c) placing the sampling subsystem in fluid communication with a detection subsystem through a fluidic transport network;
d) obtaining, by selective extraction from the sampling matrix through the fluidic transport network, a first fluid fraction that contains a different amount of the at least one biomarker relative to a second fluid fraction remaining within or subsequently extracted from the sampling matrix; and
e) generating, in the detection subsystem, the detectable signal corresponding to the at least one biomarker based on the fluid fractions.

15. The method of claim 14, further comprising:
receiving the detectable signal in a processor associated with the detection subsystem;
normalizing the detectable signal using (i) at least one physical sampling parameter obtained from the same sampling event, or (ii) at least one prior reference value associated with a subject or an external source, or (iii) a combination of (i) and (ii);
wherein the at least one physical sampling parameter is selected from sampling duration, sampling matrix deformation cycles, elution volume, and sampling matrix hydration state; and
wherein the at least one prior reference value is selected from prior measurements associated with the subject, subject-specific clinical data, population-derived statistical data, diagnostic criteria provided by the external source, and combinations of these;
and generating a diagnostic classification from the normalized detectable signal.

16. The method of claim 14, wherein the fluidic transport network comprises one or more conduits selected from passive capillary channels, pressure-driven microchannels, absorbent wicks, pump-assisted tubing, or combinations thereof.

17. The method of claim 14, wherein during an initial application of the transport mechanism, passive capillary channels of the fluidic transport network preferentially extract the at least one biomarker from a surface-proximal capture region of the sampling matrix.

18. The method of claim 16, wherein the fluidic transport network includes passive capillary channels that comprise a defined flow-resistance geometry configured to regulate a rate of extraction of the at least one biomarker from at least one capture region of the sampling matrix.

19. The method of claim 14, wherein sequential changes in distinct physicochemical parameters differentially extract the at least one biomarker from the sampling matrix.

20. The method of claim 14, wherein the fluidic transport network operates without user handling of fluid during transfer of the biomarker-enriched fluid fraction.
